(19) United States
(12) Patent Application Publication
(10) Pub. No.: US 2006/0154306 A1
(43) Pub. Date: Jul. 13, 2006

(54) Methods detecting, characterising and monitoring hydrocarbon reservoirs

(76) Inventors: Hans Kristian Kotlar, Heimdal (NO); Hakon Gunnar Rueslatten, Trondheim (NO); Martin Valeur Ramstad, Tronheim (NO); Odd Gunnar Brakstad, Trondheim (NO)

Correspondence Address: NEEDLE & ROSENBERG, P.C., SUITE 1000, 999 PEACHTREE STREET, ATLANTA, GA, 30309-3915, US

(21) Appl. No.: 10/526,512

(22) PCT Filed: Sep. 5, 2003
(86) PCT No.: PCT/GB03/03864

§ 371 Date: Dec. 15, 2005

(30) Foreign Application Priority Data
Sep. 6, 2002 (GB) 0220798.3

(51) Int. Cl. C12Q 1/68; G01N 33/567

(52) U.S. Cl. 435/7.2; 435/6

(57) ABSTRACT

The present invention relates to a method of detecting, characterizing or monitoring a hydrocarbon source, which method comprises the genotypic analysis of a sample for the presence of one or more thermophilic or extemophilic microorganisms and in particular to the generation of microbiological profiles of samples and to the comparison of these profiles with profiles from reference samples. Also provided are oligonucleotides for use in said method.

The present invention is in the field of oil drilling, exploration and production, in particular, relating to new and improved methods for identifying and monitoring sources of hydrocarbon (known as hydrocarbon zones) in the earth's crust (typically confined in sedimentary rocks). Hydrocarbon (typically petroleum, i.e. natural gas or oil e.g. crude oil) exploration is very expensive and many geophysical techniques and petrophysical techniques are used to produce data which is used to pinpoint suitable areas for explorative drilling, and to identify geological formations that may contain accumulations of petroleum.

Characterisation and identification of oil and formation water are, in general, carried out by petrophysical measurements in the well or by geochemical analysis of fluid samples, but the analytical results may be difficult to interpret in terms of useful information regarding important exploration or production issues, so there is a continued need for improved or alternative methods of identifying and characterising hydrocarbons in the geological formations and reservoirs. The present invention will improve the interpretation by introducing a new and independent data set that may also provide information regarding the migration paths of petroleum from the source rock to the reservoir(s) and function as an inherent tracer in the fluids in the production phase.

Exploration wells are drilled in areas of interest and it is desirable to use these wells to obtain information about the site, in particular whether hydrocarbons are present and at what depths. Core samples obtained from these wells can be analysed for the presence of hydrocarbons and at present this is typically done using petrophysical or geochemical techniques. However the results of these analyses are often difficult to interpret so there is a continuing need for improved or alternative methods of identifying hydrocarbon zones.

In addition, present techniques which might be used determine whether a given hydrocarbon sample is from a certain reservoir are not reliable. Tracers are difficult to place in different reservoir formations and geochemical techniques based on, for example, a chromatographic ‘fingerprint’ of the oils in a sample may not be specific enough as using this technique it is difficult to differentiate between similar oils e.g. 2 light paraffinic oils, both rich in n-alkanes.

U.S. Pat. No. 5,055,397 describes a geomicrobiological exploration method that uses “pathfinder” substances to identify regions of potential interest for exploration for oil. The correlation of these “pathfinder” substances, such as heavy metals, with the presence of oil was used as the basis of this technique. Bacteria that show resistance to the heavy metals in question are said to represent genetically encoded tolerance of the heavy metals and thus their presence is said to indicate the likelihood of the presence of oil. Tolerance to hydrocarbons is also tested. This technique requires live samples, although culture is not always required. The technique is based on the testing of acute toxicity with a standard set of bacterial inocula, and does not relate to the testing of indigenous microbes for hydrocarbon source characterisation. The requirement for live samples means that tests cannot be repeated and therefore verified subsequent to the testing date. According to the new methods described herein, live samples are not required. In addition, the prior art tests rely on substantial amounts of microbes being present and/or the testing in parallel of samples against a range of different heavy metals or hydrocarbons.

In addition, the concentrations of the toxins used in the assays has to be carefully calibrated as ions, organic compounds and pH all affects metal toxicity. Whilst this represents one method of identifying potential oil reservoir sites it can be seen that there are significant disadvantages in this indirect method of measuring a phenotype of bacteria, i.e. their ability to overcome geochemical obstacles that are associated with the presence of petrochemicals, to provide this information. The presence of bacteria that are tolerant to heavy metals may not always be a certain indication of the presence of heavy metals in the area, and similarly, the presence of heavy metals may not always be strictly correlated with the presence of hydrocarbons.

A new and improved method to correlate microbial presence more directly with hydrocarbon zones is therefore required. Other known methods of geomicrobiological exploration rely on the key assumption that hydrocarbon reservoirs leak and the escaped hydrocarbons reach the earth surface, which may be the sea floor. Thus, the presence of methanogens at the surface is said to be an indicator of the presence of hydrocarbon zones (Rawat et al. Proc. Second Seminar on Petroliferous Basins of India Vol. 3 S. K. Biswas et al. (Eds) 1994 Indian Petroleum Publishers).

This method relies on the ability to correlate the presence of the methane-utilising bacteria with the remote reservoir. This paper particularly describes using a gas uptake-manometric technique measuring utilisation of liquid petroleum gas and propane. The correlation is only reliable when the geology of the region is relatively uncomplex as the methane may otherwise migrate significant distances laterally. This rather crude technique therefore has to be coupled with geological, geophysical and hydrochemical analysis.

In addition, this technique relies solely on the phenotypic traits of the bacteria in question, i.e. their use of hydrocarbon and this is somewhat restricting. Thus, even if for example one were to survey for the presence of genes encoding proteins that relate to hydrocarbon utilisation, this would not necessarily indicate the presence of hydrocarbons as genes for hydrocarbon utilisation/degradation are found widely in nature, even in pristine polar regions and in deep sea trenches. Methane utilising bacteria may therefore be found in areas that are not associated with hydrocarbon reservoirs. For example, methanogenic bacteria may be found in eutrophic lakes, in sea water and biotopes or natural habitats that are not directly linked to hydrocarbon reservoirs. The methanogenic bacteria detected and used are normally found at the cell surface and are not able to live at high temperatures i.e. they are not thermophiles or extremophiles.

Thus techniques that give a strong and localised indication of the presence of hydrocarbon zones and also show good reproducibility and the ability to perform direct measurement are required.

It is now proposed to exploit the relationship between the presence of certain species of bacteria and hydrocarbon zones to provide a new way of identifying, characterising and monitoring hydrocarbon zones. Certain microorganisms, typically bacteria, live exclusively or predominantly in or around hydrocarbon zones and may use oil or gas constituents as energy sources. It is possible to use these bacteria as markers for the presence of hydrocarbons or to ascertain certain properties of these hydrocarbons.

The inventors have amassed information from two different reservoirs, and different wells within these reservoirs to demonstrate the diversity of microbiological profile of these different samples. Whilst bacterial samples have previously been isolated from samples from oil fields, this is the first demonstration of a correlation between a certain microbiological profile and a sample from a particular location.

The bacteria identified in these studies are able to live at high temperatures and are thus called thermophiles or extremphiles. Thermophiles are defined as organisms that can tolerate high temperatures and grow optimally at temperatures above 50° C.

Extremophiles are organisms that grow optimally under extreme conditions, e.g. extremes of temperature, pressure, pH or salinity. The microorganisms which are analysed will preferably be indigenous to the hydrocarbon zone.

The invention therefore relates to a method of detecting, characterising or monitoring a hydrocarbon zone, which method comprises the genotypic analysis of a sample for the presence of one or more (target) thermophilic or extremophilic microorganisms. The hydrocarbon source will typically be in the sub-surface formation and the samples are thus be taken from this formation, or from natural seeps, oil reservoirs or produced oil.

The present invention is of great utility in exploration where detection and characterisation of potential hydrocarbon zones (sources) is of concern. However, it also has application when production has begun, as it enables the produced hydrocarbon and the area in and around the wells to be monitored, i.e. the production conditions to be continually or periodically observed.

In certain embodiments, the thermophilic or extremophilic microorganisms analysed will not be ‘target’ microorganisms in the sense that their identity is known before the method is begun, rather genotypic information will be gathered and used to characterise the microorganisms present in the sample. Thus, alternatively viewed, the present invention provides a method of detecting, characterising or monitoring a hydrocarbon source e.g. monitoring the production of hydrocarbons from the various pay zones, in a reservoir, which method comprises the genotypic analysis of thermophilic or extremophilic microorganisms within a sample. The nature of the sample is discussed in more detail below but is typically a sample collected from produced liquids (e.g. oil, water or oil/water mixtures) from exploration and production wells or drilling out of a cylindrical core of the reservoir rocks by using a hollow cylindrical drill pipe (even drill cuttings may be applicable for the extraction of representative oil and water samples) or the sample may be a sample collected from hydrocarbon seeps on the sea floor or on shore; in the upper soil horizon or surface.

“Petroleum” or “hydrocarbon” are used as general terms for natural gas, condensates, crude oil, heavy crude oil or solid bitumen found in porous rocks. Crude oil is a complex mixture of naturally occurring hydrocarbon compounds.

The methods of the invention can be considered ‘diagnostic’. In other words, the information obtained through analysis of the microorganisms in the sample from the exploration/production site is then used in the further management of that site. The inventors have shown that the specificity and quality of information obtained through genotypic analysis of microorganisms present is such as to allow meaningful conclusions about the hydrocarbon source to be reached. Thus in preferred embodiments of the methods of the invention, information regarding the microorganisms present is utilised in an ongoing exploration or production process.

Detection of a hydrocarbon source refers to the process of identifying the presence of a hydrocarbon source in the earth's crust i.e. the sub-surface formation. It includes the process of detecting accumulations of hydrocarbons confined in porous rocks in the Earth's crust; referred to as petroleum reservoirs. The petroleum reservoir should be located in terms of longitude, latitude and depth. Attempts at detection are commonly referred to as exploration.

Characterisation of the hydrocarbon zone according to the invention, e.g. characterisation of the oil in the reservoir, may involve the correlation of the presence of one or more target microbes with properties of the hydrocarbons in question. Characterisation may be performed as part of a detection method or it may be performed after the initial identification of a (putative) hydrocarbon zone has been made. For example the type of oil, the quantity of oil, the quality of the oil, the sulphur content of the oil, the presence of gas or the gas:oil ratio are all factors about which information is required. In addition the presence of asphaltenes, resins and NSO fraction (i.e. nitrogen, sulphur and oxygen bearing compounds) may be determined. The fact that these properties will affect the bacterial flora means that it is possible to identify relationships between the microorganism populations and these properties, as different microorganisms will thrive under different conditions, e.g. available sulphur which may be utilised by certain bacteria. Semi quantitative analysis using microarrays and quantitative analysis using diversity indices of fingerprinting patterns may also be used, in addition to the qualitative analysis. The depth of a hydrocarbon source may be identified through analysis of the microorganisms as different microorganisms live at different depths.

In this way, a pattern or fingerprint of the hydrocarbon source in terms of its microbiological population may be obtained. The pattern obtained can be compared against reference patterns derived from known, well characterised hydrocarbon zones. This may relate to reservoirs, reservoir compartments or reservoir zones. Thus, a step of comparing the information obtained regarding the microorganisms present in the sample against a database of information regarding microorganisms found in other samples, is a preferred additional step in the methods of the present invention. Knowledge about the reference samples can then be used to infer properties of the new sample.

By “monitoring” it is meant that the production from a petroleum reservoir or reservoir zone is being observed as a function of time. Produced oil (or water) samples may be analysed at regular intervals as a part of the monitoring process, and changes in its microbial population may be detected. These microbial changes may be used to infer the contribution to the oil production from the various reservoir zones in a commingled production scheme (provided that their inherent microbial populations are different) and also to infer the approach of injection water from the injector towards the production well. In this way, the dynamic reservoir behaviour can be monitored without complex geochemical analysis or by using artificial tracer compounds that has to be injected into the reservoir and produced back with the oil or water.

Thermophilic and extremophilic microbes are also associated with a number of oil reservoir problems, like corrosion, near-well plugging and reservoir souring. Many of these problems are related to the activities of sulphur-utilising microorganisms. The high content of small organic acids in many reservoirs combined with anoxic conditions, as well as concentrations of sulphate and hydrogen are important for the activities of strictly anaerobic sulphidogenic and methanogenic microbes. Microbial consortia have been found in reservoirs not associated with any microbial problems, without any injection water penetration, and with temperatures >80° C. This indicates that many hot oil reservoirs harbour an indigenous flora which is not introduced by drilling muds or injection water and which may be stimulated during specific reservoir conditions. Changes in this flora during production may be observed in order to avoid enrichment of unwanted growth with subsequent reservoir problems as described above. Such techniques may also be considered “monitoring”.

In addition, the high sensitivity of bacterial populations to changes in their growth conditions may provide an early warning system for changes that are yet to take place such as biofouling (e.g. H₂S production or plugging). For example Archaeglobus is present in sour reservoirs (i.e. those that contain free sulphur) and may thus be used as an early warning system for the presence of H₂S production. Arcobacter also may appear in hot oil reservoirs. These oxidise reduced sulphur compounds (e.g. H₂S) and their presence will also therefore act to indicate the likelihood of reservoir souring. Other bacteria are associated with other changes in the chemical condition of the reservoir. In this case, the reservoir from which the sample is taken may be the producing reservoir and the sample may be oil or hydrocarbon directly taken from the reservoir or well water etc.

Thus information on the behaviour of the hydrocarbon zone (e.g. fluid saturation) as a function of time can also be derived from the generation of data concerning the particular species or types of species that are present in the sample.

Prior art studies of hot reservoir microbes are based on culturing in specific synthetic enrichment media with subsequent isolation and characterisation of the growing organisms. However most of the extremophiles living in such areas are difficult to culture. Therefore the present invention, which provides a culture independent method represents a significant advance over the prior art.

For methods of correlating the presence of microbes with changes in oil reservoir properties, it may be desirable to have just one or two species of target microorganism, e.g. Archaeglobus.

H₂S production for example can be indicated by the presence of sulphur reducing bacteria (SRB). This is problematic for oil production as SLR can cause reservoir souring via the metabolism of sulphate. Reservoir souring and H₂S production is a problem for oil generation in that H₂S has to be removed from the production streams. In addition, hydrocarbon with a high sulphide content is less valuable than pure hydrocarbon. Deposition of iron sulphide deposits in the reservoir is also a feature of reservoir souring (biofouling). Thus, a means of detecting SRB in producing reservoirs would give a good early warning indication of the production of H₂S and enable early intervention to take place.

In addition to the production of H₂S, the microbiological profile of production reservoirs can be used to survey for the presence of bacteria known to generate high biomass with plugging potential. When plugging appears, considerable numbers of sulphate reducing microorganisms are seen. Other microorganisms may also be responsible. For example, in hot oil reservoirs, Archaeoglobus, Acinetobacter, Desulfotomaculum, Methanobacterium, Methanococcus, Methanoculleus, Nanobacterium, Petrotoga, Pseudomonas, Geothermobacter, Ralstonia, Sphingomonas, Spirochaeta, Thermoanaerobacter, Thermococcus, Thermotogales, Thermodesulfobacterium, Thermodesulforabdus, Thermodesulforamonas, Thermotoga were identified in addition to other microorganisms phylogenetically related to Erythrobacter, Arcobacter, Aquabacterium, Peptostreptococcus, Pseudomonoas and Archaeoglobus. Again early intervention will prevent expensive clean-up operations.

As well as providing an early warning of potential problems with a reservoir, the monitoring methods of the invention can be used to evaluate the progress of techniques used to overcome previously identified problems.

Any sample (e.g. from the hydrocarbon zone itself which may include a seep zone that is capable of supporting a microbial population may be used in the invention. In particular, the sample can be defined as any naturally occurring sample (e.g. earth, dust, shale or oil) containing liquid hydrocarbon and/or water that has previously been exposed to hydrocarbon. The composition of the microbial population is typically sensitive to either the presence of hydrocarbons or changes in hydrocarbon properties. The microbial population of the sample may only be indirectly sensitive to the presence of hydrocarbons. For example, analysis of samples from known oil fields indicate that rock near a hydrocarbon source or even rock which is often but not inevitably associated with a hydrocarbon source will contain certain populations of microorganisms. The sample will preferably be a core sample obtained from an exploration well or well water. The core sample may be analysed at different points, i.e. relating to different depths in the crust. In order to perform the analysis, the cores may be crushed or cut and then shaken with buffers to enable the isolation of the microbial material for analysis. The microbial material may for example be the microorganisms themselves but is preferably the nucleic acid extracted therefrom. The samples may conveniently be filtered through membrane filters, trapping microorganisms on the filter surface. The buffer used in this stage will depend on the nature of the further analysis of the sample and the nature of the sample. Different extraction methods which may be used are described in the Examples.

The sample may not be a surface sample e.g. not a sample collected from the surface or upper 2 meters of body of soil or sediment. The surface being in direct contact with air or with water. The sample is preferably collected from a core penetrating the potential hydrocarbon-bearing formations.

Particularly in the circumstances where production has begun, the sample may consist of or comprise hydrocarbons e.g. it may be an oil sample. Its analysis will allow characterisation of the hydrocarbon itself.

The exploration for petroleum (typically oil and gas confined in sedimentary rocks in the earth's crust) is very expensive, and many types of data (mainly geophysical and petrophysical data) are used to identify geological formations that may contain accumulations of petroleum.

Alternatively, the sample may be sediment from the sea floor seep zone or, for the purposes of characterising or monitoring, the sample may be an oil or petroleum sample. If oil is the sample, then the microorganisms and nucleic acids are separated from the oil using a detergent such as sodium dodecyl sulphate, washing the oil with a water based buffer or by phenol chloroform isoamyl extraction. This is usually combined with mechanical disruption generated by stirring in a suspension of small plastic beads, also possibly in the presence of nucleic acid extraction chemicals such as phenol-chloroform. Such techniques are well known in the art. Commercial kits are also available for nucleic acid extraction. If the sample is water, it is filtered through 0.22 μm filters prior to treatment. The sample may also be a mixture of oil and water or any other substance that is extracted from the production well. It may also be formation (reservoir) water, water or sediment from seep zones.

The analysis of the sample may be performed in a number of ways. It is important that the analysis is genotypic rather than phenotypic as this allows for a more accurate picture of the microbial population in the sample to be achieved. Methods that are independent of any culturing of the microorganisms in the sample are particularly preferred. A further advantage of the present method is that the samples do not even need to contain live microorganisms for the analysis to be performed. The specificity of nucleic acid/nucleic acid or protein/nucleic acid interactions means that reliable results can be achieved on only a small sample without the need for culturing. Also, there are regions within the bacterial genome which are highly conserved between bacterial species and other regions which are species specific. Therefore regions may be targeted to give information about the total bacterial population, about classes of related bacteria or about individual species.

The present invention preferably provides information regarding the microbiological profile of the sample. The ‘microbiological profile’ will vary depending on the circumstances and it may be that determining the ‘microbiological profile’ will only comprise confirming the presence or absence of a single target microorganism or class of microorganisms which is considered indicative of a certain grade of hydrocarbon or, and this may be more appropriate during production rather than exploration, of a problem with the well such as proliferation of H₂S producing microorganisms.

However, preferably, the microbiological profile will comprise qualitative (and possibly quantitative) information about more than one species or class of microorganism. This will mean a series of separate but possibly simultaneous tests will be performed on the sample, each one designed to target a certain species or class of microorganisms. The word ‘class’ is used herein in a very general sense to mean a group of microorganisms which are related functionally or taxonomically. As the analysis performed is genotypic, the groupings will typically be through sequence similarities and conserved regions, for example a probe or primer may target a range of bacteria through hybridisation to a sequence which is conserved (or substantially conserved) between them. The number of ‘tests’ performed will typically be between 2 and 20, preferably 3 or 4-8.

It is not necessary to identify every species that is contained in the sample, as it is often possible to correlate the property that is being assayed for or obtain reservoir distinguishing information by the presence of one or a few species. Usually between 2 and 10 tests will be performed, preferably 3 or 4-8 if the samples are being analysed with different probes simultaneously. The sample may be analysed for the presence of each species sequentially or simultaneously, depending on the technique used. If the samples are analysed sequentially there is no upper limit on the number of species that can be assayed for. In a preferred embodiment, the microbiological profile of the sample is determined in a single test.

‘Target microorganisms’ may include microorganisms that metabolise hydrocarbons or use them for growth, however it is preferable that the target microorganism is not identified based on the presence of genes that encode proteins that are involved in hydrocarbon metabolism or by the phenotypic ability of the microorganism to metabolize hydrocarbons. These genes encoding enzymes involved in hydrocarbon metabolism are widespread in nature and are not useful as accurate markers for the presence of hydrocarbon zones. Target microorganisms in general may include different types of bacteria (including cyanobacteria), other prokaryotes and Archaea including anaerobic methaneogens, anaerobic and aerobic sulphur-metabolizing bacteria, halophilic Archaea and moderately thermophilic Thermoplasmales.

Preferred target microorganisms are Archaea, sulphur compound utilising microorganisms, fermentative bacteria, manganese and iron reducers, methanogens and acetogens. Specific microbes which may be target microorganisms are mentioned herein.

The methods of the invention involve ‘genotypic analysis’ as opposed to phenotypic analysis, i.e. investigations of the genetic constitution of the microorganisms through direct analysis of nucleic acid rather than the physical expression of the genetic information. RNA but more preferably DNA is analysed.

In a preferred embodiment of the invention, nucleic acid based amplification methods such as PCR are used to identify and characterise the microorganisms. The use of species specific sequences of nucleic acid provides a very accurate method of microorganism typing. For example the bacterial genome has regions which show very little variation from one species to another and different regions which are highly variable and essentially species or even sub-type specific. Regions of the genome can be targeted for identification purposes which are more or less variable depending on whether it is desired to use a single microorganism species or a group of microorganisms as a marker for the presence of hydrocarbons. Similarly regions of the genome that are identical in a group of microorganisms can be used to identify that group.

Nucleic acid techniques such as PCR also display the advantage that they do not have to be performed immediately following the isolation of the sample, in contrast to techniques that rely on regrowth or testing of bacteria isolated from samples. Cultivation of bacteria is known to provide a very biased view of microbial diversity, as it is difficult to correctly predict growth conditions. It has been estimated that less than 1% of all bacteria have been identified from culture techniques. Particular species will not be identified if the culture conditions used are not optimal for their growth. For example when culture and nucleic acid techniques were compared for identification of microorganisms from hot oil reservoirs, methods that rely on the isolation and regrowth of bacterial samples identified Archaeoglobus, Acinetobacter, Desulfotomaculum, Methanobacterium, Methanococcus, Methanoculleus, Nanobacterium, Petrotoga, Pseudomonas, Ralstonia, Sphingomonas, Spirochaeta, Thermoanaerobacter, Thermococcus, Thermodesulfobacterium, Thermodeisuforabdus and Thermotoga, whereas culture-independent methods led to the identification of other dominant microbial species, phylogenetically related to Erythrobacter, Arcobacter, Aquabacterium, Peptostreptococcus, Pseudomonoas and Archaeoglobus. Culture based techniques are also time consuming and may take several days or weeks to perform. They are also not suitable for on-site analysis given the requirement for specialised culture equipment. In some instances the most comprehensive picture of microbial flora may be obtained through a combination of culture-independent and culture-dependent techniques.

When characterisation is carried out via genotyping performed using PCR or other nucleic acid based techniques it is possible to store the samples using methods that kill the microbes yet do not destroy their DNA. Examples of such methods include freezing and fixation in solvents such as ethanol, toluene, formaldehyde containing buffers or sucrose. Thus samples containing live or dead microbes are equally suitable for analysis using nucleic acid methods, in contrast to culture based techniques and the technique of U.S. Pat. No. 5,055,397.

Samples may therefore be transported or stored without losing their utility. This also allows repeated analysis of the samples to be performed, after periods of storage, for example to confirm results or to screen for microbes which may be have been identified subsequent to the isolation of the sample. In this way a more complete profile may be built up, thus increasing the accuracy of the predictions derived from this technique.

The sample may contain only a small number of individual microorganisms and the amount of time required to generate sufficient numbers to perform standard microbiological analysis may be considerable. In contrast, nucleic acid techniques such as PCR require only a small number of cells, and indeed in some instances results can successfully be achieved by performing PCR on samples containing single cells.

rDNA or ribosomal DNA is present in all living organisms and encodes ribosomal RNA (rRNA), the subunits associated with the ribosome. There are 3 types of rRNA in microbes, 5S, 16S and 23S. The sequence of these molecules and hence the DNA encoding them, is highly conserved throughout evolution as ribosomal RNA has the same function in cells of all life-forms. There are also regions of significant variability. These sequences can therefore be used to determine the evolutionary relationship between two organisms, a process known as phylogenetic analysis. The presence of a target sequence of 16S rDNA specific to a certain species may be detected by the presence of a delivered PCR product, which is not seen in other species.

Species specific differences in the sequence of 16s rDNA can therefore be utilised as markers for the identification of microorganisms. Probes that bind specifically to these target sequences may be used as PCR primers. Alternatively the probes may be used to bind to and thus specifically identify PCR products or to detect target DNA sequences directly (e.g. using FISH or microcyte® or on DNA chips). Thus in a preferred embodiment of the invention 16S rDNA sequences are used as target sequences for the identification of microorganisms.

In a preferred embodiment, the probes are not labelled but are used to perform an amplification reaction, e.g. through use of the polymerase chain reaction (PCR). Thus these probes may hybridise to target regions within the bacterial genome which are specific to a particular species or class of bacteria and result in amplification of the DNA only from target species. Alternatively the primers are designed to surround the target sequence and thus amplify a region of interest for further analysis. Performance of amplification reactions and detection of amplicons are standard techniques.

Detection through the use of amplification reactions where the probes can act as primers for an extension reaction only when hybridised to target regions is very sensitive; only a small number of bacteria, theoretically only one bacterium, is sufficient to generate a detectable signal if enough rounds of the amplification are performed. The specificity of the system can be varied due to the stringency of the probe hybridisation conditions employed.

Thus, the target DNA sequence may be the site of primer binding and therefore the probe may be a PCR primer. In the presence of the specific sequence in question, a PCR product of the desired size will be generated and can be detected using standard methods such as gel electrophoresis. This may be followed by Southern blotting, or other detection techniques in which case a further probe may be used to detect any characteristic or specific sequences that are contained in the resultant PCR product.

Probes may be designed that are not specific to a single species. It is therefore possible to detect more than one type of microbe with a single pair of general primers or using a single probe to identify sequences that hybridise to that sequence e.g. in FISH, on a microchip or using a conventional blotting technique. For example, primers referred to herein as f21ARCH and r958ARCH and f341Bac/r907Bac have been used to identify bacteria and Archaea respectively. Amplification products generated using these relatively non-specific primers may then be further analysed to yield more specific information about the microorganism populations using RFLP, DGGE, Southern blotting etc. as described herein.

By sequence specific it is meant that under the conditions used in the assay, the probe or primer only binds to the target DNA sequence and to no other DNA sequence.

The target DNA sequence is the DNA sequence that is used to characterise the particular microbe and may be unique to the species to be identified or common to a group or class of species to be identified. The sequence specific probe may recognise the target DNA sequence directly or could be used to amplify a region containing the target DNA, the product of which is then subjected to further analysis e.g. using hybridisation techniques, to characterise the specific target nucleotide sequence.

Typically the target sequence is not part of or associated with a gene whose product is involved in the metabolism of hydrocarbons.

If the sequence specific probe is a primer that recognises the target DNA sequence then the presence of an amplified product of the appropriate size, using standard gel electrophoresis will indicate that the microbe containing the target sequence in its nucleic acid (e.g. its rDNA) is present in the sample. This may optionally be further confirmed by subjecting the amplified product to hybridisation techniques such as Southern Blotting. These techniques are well known in the art. If the sequence specific probe is designed such that the region of DNA that may contain the target sequence is amplified, then the presence of an amplified product is not sufficient to identify the presence of the target microbe. Further analysis of the amplified product is required to determine whether the target sequence is indeed present. This may be performed using Southern Blotting with sequence specific probes that have been labelled e.g.. with radioactivity. These probes will bind specifically to target DNA sequences and not to other DNA sequences. A positive identification of the target sequence would therefore result from the amplification of a region of 16s rDNA that contained the correct target sequence.

Such probes may also be used directly on the DNA sample, without amplification first.

Denaturing gradient gel electrophoresis (DGGE) may be also used to analyse the PCR products. This technique is used to separate nucleic acid molecules which exhibit different melting conditions, due to variations in their sequences. The fragments, in this case the PCR products, are run on a low to high denaturant gradient acrylamide gel in which they are initially separated based on molecular weight. In the higher denaturing conditions, sequence specific DNA melting starts to occur which affects the mobility of the fragments. The mobility shift differs for different sequences and as little as 1 bp difference can cause a mobility shift. In this way species specific differences in amplified fragments of 16s rDNA can be detected in target microorganisms.

The PCR products may also be analysed using restriction fragment length polymorphisms (RFLPs). These are changes in DNA sequence that lead to a change in one or more restriction enzyme sites. So if a species specific change in a 16S rDNA sequence causes the appearance or disappearance of a restriction enzyme site then by digesting the amplified PCR product with this enzyme, followed by standard gel electrophoresis analysis to ascertain the molecular weights of the products of this digestion, the target species may be identified. If RFLP analysis is to be used then the PCR primers are designed to amplify a target DNA region containing an RFLP site. Unique RFLPs may be used to identify particular microbial species.

Any other standard technique known in the art to detect specific known nucleotide sequences in PCR products may alternatively be used. The amplified products may be sequenced directly and, as described in the present Examples, the resulting sequence information subjected to BLAST analysis.

It is preferred that the analysis is carried out using multiple probes or primers sequentially or simultaneously, in order to generate information regarding the microbiological profile of the sample. In a most preferred embodiment the analysis is performed simultaneously using multiple probes or primers, i.e. a battery of probes or primers. The battery of probes or primers will be specifically designed depending on the nature of the information that is required, i.e. the range and types of microorganisms which it is desired to detect.

Other nucleic acid based techniques, in addition to PCR or instead of, may also be used to identify bacteria present in the sample. Such techniques include those where a probe binds directly to the nucleic acid of the microbe without prior amplification of the target sequence. In this case the probes that bind to the target nucleic acid sequence are labelled to enable their detection following hybridisation. Accordingly, a further aspect of the invention relates to methods of identifying a hydrocarbon source through analysis of a sample for the presence of one or more, specific nucleic acid target sequences or markers wherein the analysis is performed by FISH, microcyte® or using a DNA microchip.

FISH (fluorescent in situ hybridisation) is a well characterised technique that is used to detect target nucleic acid sequences in individual intact cells via a system of coupled antibodies and fluorochromes. Specific nucleic acid probes of approximately 20 nucleotides in length are synthesised, incorporating labelled nucleotides. The nucleic acids may be labelled by the conjugation or addition of fluorescent molecules such as FITC, Cy5, Cy3, TRITC, Texas Red. Alternatively the probes may be labelled with an immunogenic molecule such as digoxygenin, in which case antibodies to the immunogenic molecule are used in the detection step. The antibodies might be fluorescently labelled or may be detected by other fluorescently labelled antibodies. Biotin may also be used to label the probes, and this is detected using its high affinity binding partner avidin. The probes may be targeted to DNA or preferably to rRNA.

The hybridisation is carried out by mixing the labelled probes with the sample, in which the nucleic acid has been denatured, thus providing access of the probe to the target DNA.

The probes are detected directly, using fluorescence microscopy or indirectly, following the addition of fluorescently labelled antibodies that specifically bind to the labelled probe, or to other, non-labelled antibodies that specifically bind to the labelled probe. It is possible to detect more than one type of fluorescence simultaneously, using the appropriate filters. In this way, the detection of the presence of more than one marker can be performed simultaneously.

If FISH is used, the probes will be designed to specifically hybridise with the identified target sequences for each microbial species or group of species.

FISH may be combined with other stains such as Ethidium Bromide and DAPI. This would allow the calculation of the number of microorganisms containing the target sequence relative to the total number of microorganisms present.

Microcyte® flow cytometry can also be used to detect or analyse the presence of specific bacterial species group of species. Microcyte® flow cytometry is a flow cytometry technique that is specifically adapted for microbiology and is advantageous in that it can be used with very low cell numbers. The cells are stained using fluorescently labelled rRNA probes. In this way, cells containing specific sequences can be identified and quantified.

Applying probes to a microchip and allowing sample nucleic acid to hybridise is a further way of identifying sequences homologous to (or sufficiently homologous to bind to) these probes. The probes to which sample DNA hybridises can be readily identified if the sample nucleic acid has been labelled. The probes are attached to the chip at known locations and the location of the signal indicates to which probe the nucleic acid has bound. The sample nucleic acid for example may for example be genomic DNA, total RNA, mRNA, cRNA, cDNA, PCR product and each of these or any other nucleic acid may be labelled prior to contacting with the chip according to standard techniques.

From all the above discussions, it is clear that the methods of the invention typically comprise a step wherein an oligonucleotide binds to a region of nucleic acid within the nucleic acid of or derived from the target microorganisms. Thus the “genotypic analysis” preferably comprises contacting the sample with one or more species of oligonucleotide, said oligonucleotides being designed to hybridise with or adjacent to characterising sequences within the nucleic acid of target microorganisms. The term oligonucleotide encompasses probes (e.g.,labelled probes) and primers for amplification or sequencing-by-synthesis methods. Unless otherwise clear from the context, the terms probes and primers may be used interchangeably herein. The ‘characterising sequences’ are those target sequences described above which may be species or class specific. As previously mentioned, where an amplification reaction is performed, primers may hybridise adjacent to (i.e. one on each side of) a characterising sequence. This also presents the opportunity for two layers of specificity if selective primers are chosen and then a further hybridisation reaction to analyse the amplified sequence is also performed.

As preferred variants of all of the above methods, a number of different probes or primers which hybridise to target sequences within different bacterial species may be used simultaneously, which will give a pattern of the bacterial flora present in a sample i.e. a microbiological profile of the sample. The pattern may be visualised following gel electrophoresis in the case that it is produced by PCR. Alternatively, as mentioned above, FISH, microcyte or other nucleic acid based technologies that permit the visualisation of multiple probes in a single sample may be used. Such a system which provides more detailed information about the bacterial species present may give a good indication of the conditions in the environment from which the sample was taken. The pattern obtained can be compared against reference patterns derived from samples from known hydrocarbon zones.

The analysis (preferably the simultaneous analysis) of multiple species to generate a microbiological profile therefore represents a preferred embodiment of the invention.

In preferred embodiments, the methods of the present invention (more particularly the exploratitive methods of the invention) result in the generation of a microbiological profile for a given sample. Qualitative information about the presence or absence of 2 or more types (i.e. species or groups/classes), preferably 3 or 4 or more, e.g. 3 or 4-8 different types. This results in the generation of a pattern which can be compared, manually or through computer analysis with reference samples to provide a good indication as to the hydrocarbon potential of the sample.

Thus, in a preferred embodiment the present invention provides a method of evaluating a sample (e.g. from a sub-surface formation, a seep sample or an oil reservoir sample) by the genotypic detection of a plurality of different target thermophilic or extremophilic microorganisms, the generation of a microbiological profile for said sample and optionally comparison of said profile with one or more reference profiles. The evaluation may be to determine the hydrocarbon potential of, or the type of oil that may be present in, the area or region from which the sample was taken or it may be part of an analysis or monitoring programme performed on a known reservoir or well in a reservoir e.g. a production phase sample. The term ‘sub-surface formation’ is the term generally used for the region of the earth's crust in which hydrocarbons are located.

The evaluation may alternatively provide information with respect to migration routes, reservoir fill/spill evaluations and reservoir depths.

A further aspect of the invention relates to the surveillance and analysis of oil from different formations in a commingled production in the production phase, i.e. where the produced oil is derived from two or more reservoirs. This will occur if there is reservoir continuity. Alternatively, commingling of oil from different reservoirs may also occur if there are fault lines. The technique may also be used to detect whether two oil samples drawn from different wells are in fact derived from a single continuous reservoir or from separate reservoirs. In the production phase, the evaluation can be used to identify the producing zones in a commingled production scheme and monitoring the approach of injection water towards the production well. It is critical to oil field management that the structure of the reservoirs should be understood.

It will be appreciated that once the individual microorganism profiles been established for the two or more reservoirs or producing zones, it will be straightforward to determine during production if the oil product is in fact a mixed product from the two reservoirs by analysing its microorganism content. For example if species A, B and D are found in one reservoir or producing zone, and species B, C and E are found in a second producing zone, then the presence of only species A, B and D indicates that the oil is coming from the first reservoir or producing zone, and action can be taken to shut off or modify production from the second (B, C and E containing) zone. Quantitative analysis may indicate the relative contributions of one or more producing zones to a collected hydrocarbon.

In the art commingling has been investigated by pressure testing and mapping or by injecting tracers such as radioactive tracers or organic fluorescent substances into the different reservoirs. Suitable tracers may be pressure testing is very expensive and also not always possible. Injection of tracers into the reservoir formation is a very difficult procedure and also not always possible. It may be possible to evaluate natural existing components in the oil or water which may be specific for a particular reservoir in order to determine the source of commingled products, however such reservoir specific components may not always exist.

A method that can be used for any oil field is therefore required which does not require production to be ceased and which can be used for continuous monitoring.

By analysing the microbiological profile of the produced oil or water it will be possible to correlate either the presence of certain microbes, or the overall pattern of microbes with the source of the oil.

Analogously, in the exploration phase, migration routes can be studied, by comparing the microbiological profiles of samples collected at different locations.

Reservoir fill/spill evaluations may also be performed in this way. If for example oil from one reservoir has spilt over into a second reservoir then the presence of new microbiological species in the other reservoir may indicate the origin of the spilt oil.

Microorganisms that may be identified and whose presence is correlated with the characteristics and presence of hydrocarbons include representatives of bacteria and Archaea. Archaeoglobus, Acinetobacter, Bajacaliformiensis, Desulfotomaculum, Methanobacterium, Methanococcus, Methanoculleus, Nanobacterium, Petrotoga Pseudomonas, Ralstonia, Sphingomonas, Spirochaeta, Thermoanaerobacter, Thermococcus, Thermodesulfobacterium, Thermodesulforabdus, Thermotoga, Acidoaminococcus, Aminobacterium, Halomonas, Desulfomicrobium and Methylobacterium, have all been identified in oil fields and may therefore constitute target microorganisms.

The ability of many Archaea to survive in the conditions found in production and explorative wells make them particularly suitable as target microorganisms. Generally, Archaea can be identified by their cell wall which lacks a peptidoglycan skeleton and by their cytoplasmic membrane which contains glycerol ethers with C₂₀ (phytanyl) and C₄₀ (biphytanyl) alkyl isoprenoids in place of the fatty acid glycerol esters. In addition, the DNA-dependent RNA polymerases of Archaea differ from those of Bacteria in that they consist of more than four subunits and are resistant to the antibiotics rifampicin and streptolydigin.

These and other microbes are specially adapted to live under the conditions found in oil wells. For example they are thermophiles or extremophiles. They may also be methanogenic, sulphur using, or able to live at great depths.

A further use of the evaluation and characterisation techniques described herein is to evaluate reservoir depths. There is a direct correlation between temperature and depth. There is also a direct correlation between temperature and the ability of microorganisms to grow and therefore the presence of certain microorganisms provides information directly as to the temperature and indirectly as to the depth of the sample.

As it is not necessary that the sample contains live microorganisms, it is also possible to derive information concerning past properties of the oil, as long as the nucleic acid of the microorganism(s) that provide this information is preserved in the sample.

As the properties of oil may be changed by the presence of one or more microorganisms, through the alteration and/or metabolism of components of the oil, (oil degradation) it is useful to know whether the current properties of an oil sample are inherent properties or whether they have been brought about by oil degradation. The presence of certain microorganisms known to be associated with biodegradation can be used either to diagnose biodegradation and could allow early intervention with suitable biocides.

The invention also relates to probes or primers for use in the method of analysis. For example PCR primers that recognise species specific 16s rDNA sequences and thus generate species or class specific amplification products form part of the invention. As mentioned above, the PCR primers may also be designed to specifically amplify DNA sequences containing the target regions of 16s rDNA that contains a species specific sequence that can then be identified using RFLP analysis or hybridisation analysis techniques that are known in the art.

The probes may alternatively be used to bind directly to nucleic acid prepared from or present in the sample without prior amplification.

Various new probes are described herein: which are suitable for identifying individual species or groups of species. Such probes themselves constitute a further aspect of the present invention.

The probes or primers may recognise a single species or a class of related species. For example there are primers/probes that will identify bacteria; f341Bac and 907Bac, 341fBac: 5′-CCT-ACG-GGA-GGC-AGC-AG-3′ (SEQ ID NO: 1) (forward primer) 907rBac: 5′-CCC-CGT-CAA-TTC-CTT-TGA-GTT-3′ (SEQ ID NO:2) (reverse primer), which give an expected PCR product of 567 bp. There are also primers/probes that identify Archaea, f21ARCH and r958ARCH 21fARCH: 5′-TTC-CGG-TTG-ATC-CCG-CCG-GA-3′ (SEQ ID NO:3) (forward primer) 958rARCH: 5′-CCC-GGC-GTT-GAA-TTC-AAT-T-3′ (SEQ ID NO: 4) (reverse primer) which give an expected PCR product of 938bp (Teske et al. 1996 Appl. Environ. Microbiol. 62: 1405-1415, DeLong et al. 1992, PNAS USA 89(1): 5685-9). There are also primers that recognise groups of sulphur reducing bacteria (Desulphovibrio) e.g. f341Bac and r687SRB (r687SRB: TACGGATTCACTCCT) (SEQ ID NO: 5). Probes/primers that recognise Desulfovibrio and Desulfobulbus (f385SRB: 5′-CGG-CGT-CGC-TGC-GTC-AGG-3′ (SEQ ID NO: 6) and r907BAC) and probes/primers that recognise Desulfobacter and Desulfobacterium (f341Bac and r804SRB: 5′-CAA-CGT-TTA-CTG-CGT-GGA-3′) (SEQ ID NO: 7). These may be used for PCR or for direct hybridsation techniques.

Probes that are suitable for use in FISH, for application to a microchip or for other hybridisation techniques include EUB338: 5′-GCTGCCTCCCGTAGGAGT-3′ (SEQ ID NO:. 8) (against bacteria generally); ARCH915: 5′-GTGCTCCCCCGCCAATTCCT-3′ (SEQ ID NO: 9) (against all Archaea generally); NON338: 5′-ACTCCTACGGGAGGCAGC-3′ (SEQ. ID NO: 10) (negative to all bacteria and Archaea); ARGLO605: 5′-GCCTCTCCCGGTCCCTAG-3′ (SEQ ID NO:11) (against all registered Archaeoglobus) THERSI672: 5′-CCCTACACCAGCAGTTCC-3′ (SEQ ID NO: 12)(against all registered Thermotogales). Ery368: 5′-CCAGTATTCTAGCCATCC-3′ (SEQ ID NO: 13) (for all registred Erythrobacter and Erythromicrobium); ARC293: 5′-TCCATCTACCTCTCCCAY(C or T)-3′ (SEQ ID NO: 14)(for all registered Arcobacter).

GCGGCTWCCTGGACCACCGATACT, (Desulforomonas; Geothermobacter SEQ ID NO: 15), GGCGGTGAAATTTTGCAGCTCA (Baceroides, SEQ ID NO: 16) and GTAGGCGGAATTTGTGGTGTAGC (SEQ ID NO: 17) are three further probes that may be used.

554 ARCHI 5′-TTA-GGC-CCA-ATA-ATC-MTC-CT-3′, (SEQ ID NO: 18) is a probe that has been used for Southern Blotting and may be used in any direct hybridisation technique to recognise Crenarchaeota (group I Archaea). 544 ARCHII 5′-TTA-GGC-CCA-ATA-AAA-KCG-AC-3′, (SEQ ID NO: 19) is a further probe that has been used for Southern Blotting and may be used in any direct hybridisation technique to recognise Euxyarchaeota (Group II Archaea).

Thus the novel probes described above or their complements represent a further aspect of the invention. Also encompassed within this aspect of the invention are functionally equivalent variants of these novel probes, i.e. probes of equivalent specificity but of different lengths or with modified sequences. Typically these variant probes will be no less than 6 nucleotides shorter, preferably no less than 3 nucleotides shorter and no more than 10 nucleotides, preferably no more than 5 nucleotides longer than the specified probes listed above. Variant probes/oligonucleotides will typically have at least 80%, preferably at least 90%, e.g. at least 95% sequence identity with the specific probes listed above.

The probes may be designed to bind directly to the characterising sequence, e.g. for use in FISH or for direct hybridisation e.g. on chips using conventional blotting techniques. The probe may be labelled or tagged with a fluorescent marker, a radioactive marker or an antigenic label.

The sequence specific probes may identify sequences that are specific to microbes that are associated with a particular oil field area.

Combinations of probes or primers may be used in the method of the invention. Probe mixtures comprising probes to identify more than one microorganism simultaneously, or to provide a microbiological profile therefore form a further aspect of the invention.

The conditions under which the analysis is performed may also be used to provide different levels of specificity. For example, changing the buffer composition in which hybridisation of the sequence specific probe hybridises may be modified to change the stringency. Similarly the temperature at which the annealing step of the PCR occurs may be modified. Such modifications are well known in the art.

Also indicated within the scope of the invention are kits for performing the methods of the invention. Such kits comprise one or more oligonucleotides designed to hybridise to or adjacent to characterising sequences and may comprise pairs of primers for performing amplification and/or single probes directed towards microorganisms, these single probes may be labelled. The selection of primers being such that a microbiological profile of the sample is attained which may be used in the evaluation of the sample for indicators of a hydrocarbon source, for the presence of markers of production problems, etc. In a preferred kit, the probes are attached to a solid support, e.g. a microchip. In this case, the probes will be designed to hybridise to characterising sequences rather than take part in an amplification reaction.

As well as oligonucleotides, the kits will typically comprise suitable buffers for use in the test procedure, e.g. a lysis buffer and other buffers and components for use in an amplification reaction e.g. DNA polymerase (particularly Taq DNA polymerase) and nucleotides for incorporation into the amplified products.

Chips to perform hybridisation analysis comprise another aspect of the invention. The chips preferably contain the probes described above. More preferably a bacteria specific probe (e.g. SEQ ID NO: 8) plus an archeal specific probe (e.g. with SEQ ID NO:9) plus one or more of the probes selected from the group comprising SEQ ID NOs: 8 to 17 or functionally equivalent variants of these probes are attached. Preferably the chip contains 2 or more, 4 or more or 6 or more oligonucleotide probes.

The invention will now be described further with reference to the following non-limiting Examples and the figures in which:

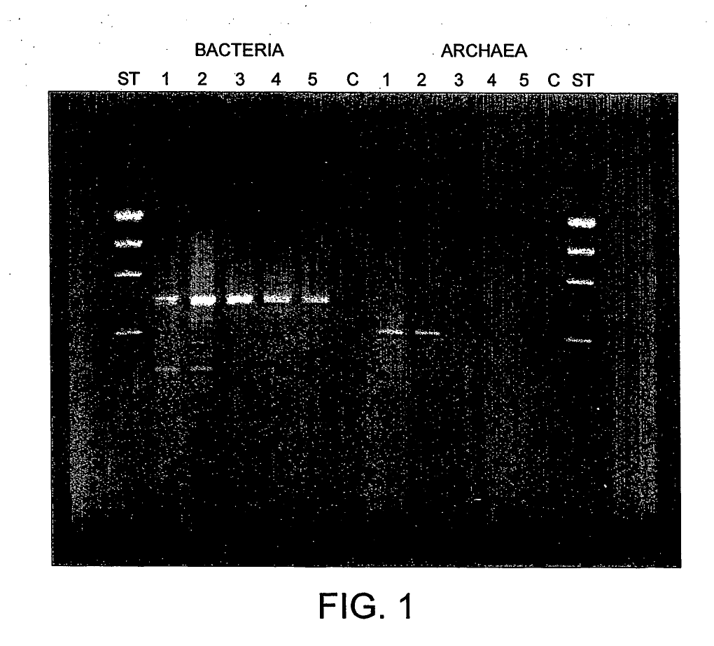

FIG. 1 shows results from agarose gel electrophoresis of PCR products amplified with primer sets defining Bacteria and Archaea, respectively. The samples are reservoir 1 well 1 (1), reservoir 1 well 2 (2), reservoir 2 well 1 (3), reservoir 2 well 2 (4), reservoir 2 well 2 washed interphase (5);

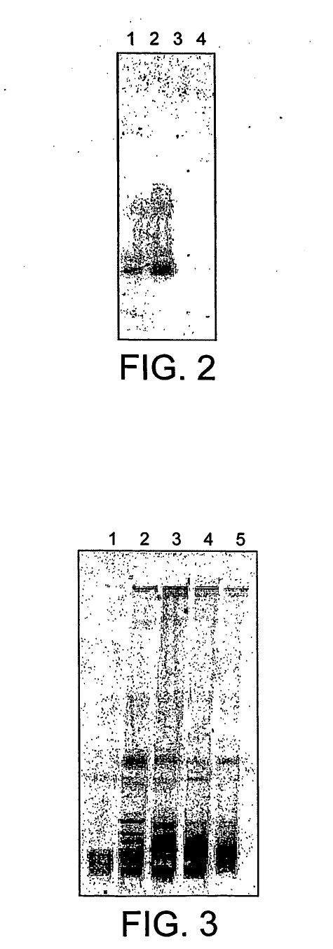

FIG. 2 is a southern blot analysis of PCR products from reservoir samples after DNA hybridisation with the biotinylated probe 385SRB defining δ-subdivision bacteria. The samples are reservoir 1 well 1 (1), reservoir 1 well 2 (2), reservoir 2 well 1 (3), reservoir 2 well 2 (4);

FIG. 3 shows results from DGGE of PCR products amplified with primer sets defining Bacteria. The samples are reservoir 1 well 1 (1), reservoir 1 well-2 (2), reservoir 2 well 1 (3), reservoir 2-well 2 (4) and reservoir 2, well 2 washed interphase (5);

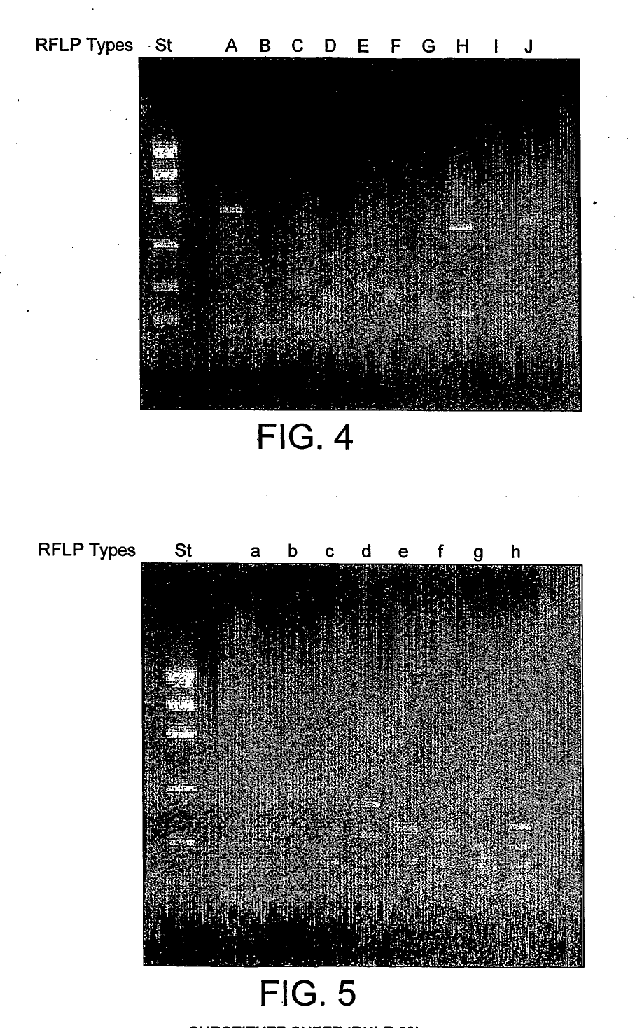

FIG. 4 shows HaeIII RFLP types of cloned PCR products from DNA extracted from the two reservoirs;

FIG. 5 shows RsaI RFLP types of cloned PCR products from DNA extracted from the two reservoirs;

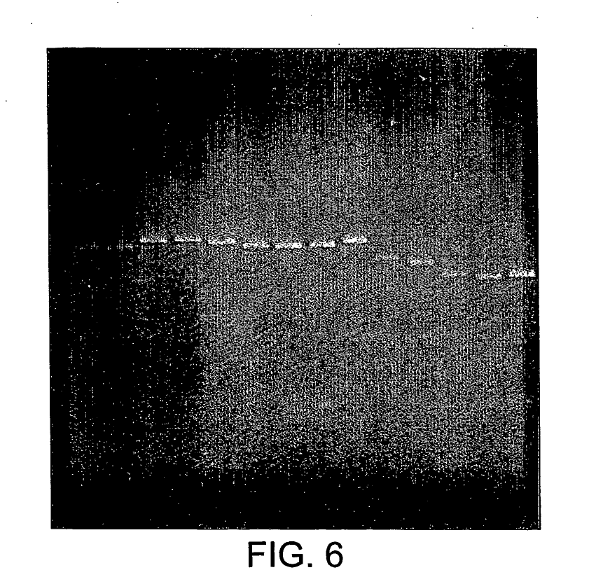

FIG. 6 shows differences in band migration within clones characterised as HaeIII RFLP type A;

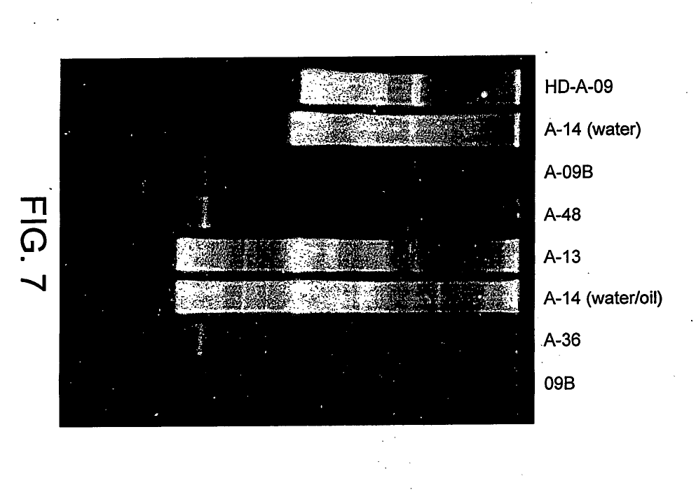

FIG. 7 provides DGGE results of fines received 17.8.01 amplified with primers defining Bacteria.

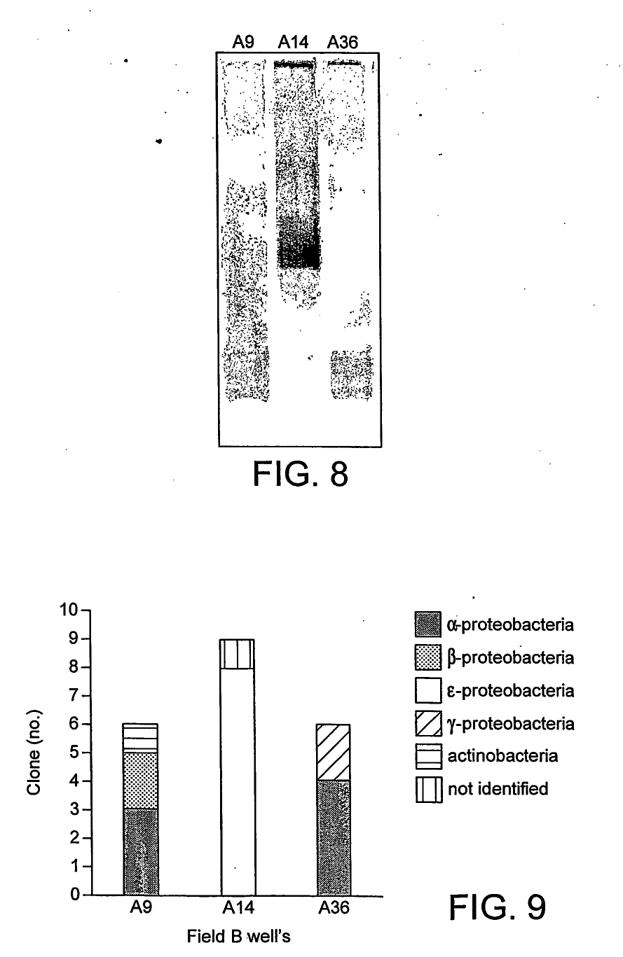

FIG. 8 provides DGGE results of samples from reservoir 2 amplified with primers defining bacteria.

FIG. 9 shows the numbers of clones of various bacterial types found in 3 wells in field 2.

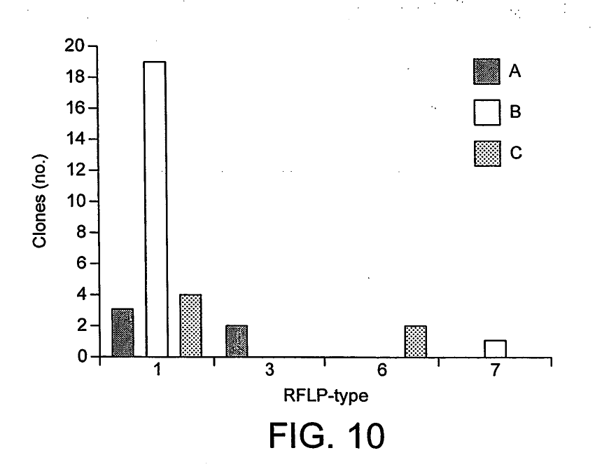

FIG. 10 shows RFLP analysis of Archael samples.

### EXAMPLE 1

Identification of Archae and Bacteria indigenous to oil fields.

1. Materials and Methods

1.1 Sample Collection and Processing

1.1.1 Sampling

Sterilized 5-L sample glass bottles (Schott) with 5 ml of 0.1% (wt/vol) resazurin reducing agent (Sigma Chemical Co., St. Louis, Mass., USA) per bottle were shipped to the offshore fields. Samples were collected as raw production fluid (oil/water) from the production flow-line before produced water separation. Bottles were completely filled. The top oil layer sealed the water from the atmosphere, eliminating oxygenation of the water phases.

Samples were sent onshore in sealed Al-boxes (transport periods 1-2 weeks). During transportation the boxes were kept at ambient temperature.

No traces of oxygen were detected in the water phases when the samples arrived onshore.

1.1.2 Filtration of water samples

After arrival at the laboratory the oil and water phases of the production fluid samples were immediately heated (70° C.; 20 minutes) and the hot phases separated in 2 L separating funnels (2 L). Microbial biomass from 1-2 L water was collected by filtration through 0.22 μm Sterivex GV filters (Millipore Corp., Bedford, Mass., U.S.A) connected to Millex AP prefilters (Millipore). After filtration each Sterivex filter was filled with a 2 μl lysis buffer (50 mM Tris-HCl, pH 8.0; 40 mM EDTA; 750 mM sucrose) and stored at −20° C. until DNA extraction.

One of the samples was highly emulsified, resulting in only a small water phase. This emulsion phase was washed with a sterile buffer solution, phase separated, and filtered (approximately 1 L buffer phase) as described above.

1.2 Cell Counts

Cell numbers in water phases of the production fluids were enumerated immediately after the arrival at the laboratory by epifluorescence microscopy. Samples were pre-filtered (Millex AP, Millipore), and 10.0 ml filtrates centrifuged (6000× g; 10 minutes to remove coarse particles and residual oil droplets. The fluorochrome 4′6-diamino-2-phenylindol (DAPI; 0.6 μg/ml) was applied to the supernatants (10 ml), and the samples incubated at room temperature (10 minutes), followed by filtration through 0.22 μm black polycarbonate filters (Millipore). The filters were mounted in a fluorescence microscope (Leitz Dialux with Ploemopak fluorescence unit and UV-filter A and equipped with a Leica DC 100 digital camera) with immersion oil. Fluorescent cells were enumerated with 1250× magnification.

1.3 Polymerase Chain Reaction (pcr) Amplification of Reservoir Samples

1.3.1 Nucleic Acid Extraction and Quantification

The frozen Sterivex filters with microbial communities were thawed and lysed directly on the filters. The lysis was performed by incubation of each filter with 2 μg lysozyme (Sigma; from a 20 mg/ml stock solution; 37° for 30 minutes). The mixtures were then incubated at 55° C. for 2 hours with 1 μg Proteinase K (Sigma; from a 20 mg/ml stock solution), and 1% (wt/vol) sodium dodecyl sulphate (SDS; BioRad Labs, Richmont, Calif., USA) from a 20% stock solution.

The lysates were transferred to sterile tubes, the Sterivex filters washed with lysis buffer (55° C.; 10, minutes), and the lysates from each filter pooled. The lysates were extracted with hot phenol-chlorophorm-isoamylalcohol (25:24:1) according to standard procedures (Sambrook and Russel, 2001). Briefly, each lysate (3 ml) was mixed with 6 ml hot (60° C.) Tris-HCl buffered phenol-chlorophorm-isoamyl-alcohol (pH 8.0), vigorously shaken, maintained hot for 5 minutes, then cooled on ice, followed by phase separation by centrifugation (4000× g; 5 minutes at 4° C.; Jouan Model 1812, Saint Nazaire, France). Sodium acetate (0.2 volume of 10 M solutions) was applied to each water phase, and these were re-extracted with 5 ml Tris-HCl buffered phenol-chlorophorm-isoamylalcohol, followed by centrifugation. The water phases were then extracted with 5 ml hot (60° C.) chlorophorm-isoamylalcohol (24:1), and centrifuged as described above. The extracted water phases were precipitated by 2.5 volumes of 96% ethanol (−20° C.; 3 hours), the precipitates pelleted by centrifugation (4000× g), and the pellets washed with 75% ethanol. The pellets were dried (N₂) after re-centrifugation and dissolved in 100 μl sterile ultra-pure water (Biochrom AG, Berlin, Germany). The nucleic acid extracts were frozen (−20° C.).

Extracted nucleic acids were semi-quantified with an ethidium bromide method (Sambrook and Russel, 2001). Nucleic acids (2 μl) were spotted on a UV transilluminator table (BioRad) wrapped with plastic film (“GladPak”), and 2 μl ethidium bromide (10 mg/ml in TE buffer, pH 8.0) were applied to each spot. The spots were photographed under UV-illumination and concentrations determined by intensity comparison to a standard series of salmon DNA (Sigma) in the range 100-500 ng DNA per spot.

1.3.2 Oligonucleotides and PCR Reagents

1.3.2.1 Oligonucleotides for PCR Amplification

Oligonucleotide primers were prepared specific for Bacteria and Archaea (Teske et al., 1996; DeLong, PNAS USA 89(1):5685-9, 1992):

Bacteria
341fBac: 5′-CCT-ACG-GGA-GGC-AGC-AG-3′
(forward primer SEQ ID NO: 1)
907rBac: 5′-CCC-CGT-CAA-TTC-CTT-TGA-GTT-3′
(reverse primer SEQ ID NO: 2)
Expected PCR product: 567 bp
Archaea
21fARCH: 5′-TTC-CGG-TTG-ATC-CCG-CCG-GA-3′
(forward primer SEQ ID NO: 3)
958rARCH: 5′-CCC-GGC-GTT-GAA-TTC-AAT-T-3′
(reverse primer SEQ ID NO: 4)
Expected PCR product: 938 bp

The primers were synthesized by EuroGentec, Seraing, Belgium. The primers were diluted in sterile water at concentrations of 50 μM, distributed in 50 μl aliquots and stored at −20° C.

1.3.2.2 Biotinylated Oligonucleotides

A number of biotinylated DNA oligonucleotides (5′- and 3′-labelled) were prepared (Teske et.al., 1996; Massana et. al., 1997) for Southern blotting analysis of PCR products:

δ subdivision/gram-positive bacteria (included
Desulffovibrio and Desulfobulbus)
385 SRB:
Biotin-5′-CGG-CGT-CGC-TGC-GTC-AGG-3′-Biotin
Hybridization temperature: 50° C. (SEQ ID NO: 6)
Desulfobacter and Desulfobacteriuxn
804 SRB:
Biotin-5′-CAA-CGT-TTA-CTG-CGT-GGA-3′-Biotin
Hybrization temperature: 40° C. (SEQ ID NO: 7)
Crenarchaeota (Group I Archaea)
554 ARCH-I:
Biotin-5′-TTA-GGC-CCA-ATA-ATC-MTC-CT-3′-Biotin
Hybrization temperature: 40° C. (SEQ ID NO: 18)
Euryarchaeota (Group II Archaea)
554 ARCH-II:
Biotin-5′-TTA-GGC-CCA-ATA-AAA-KCG-AC-3′-Biotin
Hybrization temperature: 40° C. (SEQ ID NO: 19)

All biotinylated primers were synthesized by EuroGentec, Seraing, Belgium. The primers were diluted in sterile water at concentrations of 50 μM, distributed in 50 μl aliquotes and stored at −20° C.

1.3.2.3 Deoxynucleotides

Stock solutions of deoxynucleotides (d′NTP) were prepared by diluting 100 mM of the d′NTPs 2′-deoxyadenosine 5′-triphosphate (d′ATP), 2′-deoxythymidine 5′-triphosphate (d′TTP), 2′-deoxyguanosine 5′-triphosphate (d′GTP) and 2′-deoxycytidine 5′-triphosphate (d′CTP) (Amersham Pharmacia Biotech, Piscataway, N.J., U.S.A). Each d′NTP (100 μl) was diluted in sterile water (600 μl) , resulting in final concentrations of 10 mM of each d′NTP. Solutions were distributed in 50 μl aliquots and stored at −20° C.

1.3.3 Touchdown PCR

Extracted DNA (see above) or lysed microbial cell suspensions were used as DNA template for PCR amplification. When lysed cell suspensions were used broth cultures were diluted in sterile water (10⁻²) or colonies from agar plates suspended in sterile water, followed by heating (100° C.) in 10 minutes.

A PCR mix of 100 μl mix consisted of 20 μl d′NTP (10 mM), 10 μl forward primer (50 μM), 10 μl reverse primer (50 μM), 55 μl sterile water and 5 μl AmpliTaq DNA polymerase (Perkin Elmer Roche Molecular Systems, Branchburg, N.J., U.S.A).

DNA template (1-10 μl) was diluted in 10 μl [10×] PCR buffer with 15 μM MgCl₂ (Perkin Elmer Roche) and with sterile water to a final volume of 90 μl. The mixture was heated (95° C.) in 5-10 minutes on a heating block. A PCR mix of 10 μl was applied to each sample when the samples were still in the heating block (95° C.) and the samples were immediately transferred to a DNA Thermal Cycler (iCycler, BioRad).

PCR was run as a touchdown method to reduce the generation of spurious by-products, and with the following sequence cycles:
Denaturation: 95° C. for 1 minute
Primer annealing: 65-55° C. for 1 minute
DNA synthesis (primer extension): 72° C. for 3 minutes
Number of cycles: 35

During the first 10 cycles the annealing temperature was gradually reduced from 65 to 55° C. with 1° C. for each cycle during the first 10 cycles, followed by 25 cycles with annealing temperature of 55° C. The PCR runs were terminated by 72° C. for 15 minutes before cooling to 4° C.

1.3.4 Agarose Gel Electrophoresis

1.3.4.1 Analytical Electrophoresis

PCR products were analysed by horizontal agarose gel electrophoresis. Samples (27 μl) were mixed with [10×] gel-loading TBE buffer (3 μl) (0.9 M Tris, 0.9 M borate, 20 mM EDTA, pH 8.3, 50% (v/v) glycerol, 0.25% (w/v) bromophenol blue). A Low DNA Mass Ladder (Gibco BRL, Paisley, UK) was used as standard, 12 μl standard in 3 μl [10×] gel-loading TBE buffer.

Gels were prepared by heating agarose (2.0 g; Sigma) in 160 ml [0.5×] TBE (0.045 M Tris, 0.045 M borate, 1 mM EDTA, pH 8.3) in a microwave oven (4 minutes), followed by cooling to 50° C. in water bath. Ethidium bromide (16 μl) from a stock solution (10 mg/l ethidium bromide in sterile water) was applied to the agarose, and the melted gel was casted horizontally in a plastic tray (open ends of the tray sealed) with a comb of 15-well or 20-wells in the electrophoresis apparatus (BioRad). The gel was set at room temperature for 20 minutes, submerged in [0.5×] TBE buffer, and the comb and seals carefully removed.

Prepared samples and standard (see above) were applied to the submerged gel wells (20 μl sample and 10 μl standard) and electrophoris run with constant voltage (150 V) for 1.5-2 hours at room temperature. Gel documentation was performed over a UV-transilluminator table (BioRad). The-gels were photographed by black-white Polaroid film (0.1-0.5 second exposure time), or by digital camara (GelDoc, 2000, BioRad).

1.3.4.2 Preparative Electrophoresis

Preparative agarose gel electrophoresis was performed basically as described above for the analytical approach, except that a low-melt agarose (Sigma) was used. Agarose (1.3 g) was melted in 160 ml [0.5×] TBE buffer or [1×] TAE buffer (0.04 M Tris-acetate, pH 8.0; 1 mM EDTA), the gel solution cooled to 35-50°, ethidium bromide applied, and the horizontal gel as described above, except that the set temperature was 4-5° C. Samples were applied as described above, and electrophoresis run at 100 V constant voltage for 1.5-2 hours.

After electrophoresis the gels were photographed and selected DNA bands cut out from the agarose with a sterile scalpel and transferred to microcentrifuge tubes. Before further processing the agarose slices were pelleted with a brief centrifugation and melted at 65° C. for 15 minutes. The samples were maintained melted at 35-37° C.

1.3.5 Southern Blotting and Hybridization

1.3.5.1 Blotting

By Southern blotting agarose gel electrophoresis of PCR products were performed as described above (analytical approach), except that no ethidium bromide was added to the gel. DNA was transferred from the gel to Hybond N+ membranes (Amersham Pharmacia) by diffusion blotting.

After electrophoresis the gel was soaked in 10 volumes of denaturation solution (0.5 M NaOH, 1.5 M NaCl) for 2×20 minutes (slow agitation), followed by neutralization (1 M ammonium acetate) for 2×15 minutes. The gel was then trimmed and placed on a glass plate with chromatographic paper (3 mm Chr, Whatman, Maidstone, U.K.) soaked in 1 M ammonium acetate. The ends of the chromatographic paper was placed into a bath of transfer buffer (0.2 M ammonium acetate). The gel was surrounded by a thin plastic film (“GladPak”) to prevent transfer buffer evaporation. The Hybond N+ transfer membrane was soaked in 0.2 M ammonium acetate and placed tightly on the top of the gel. Several layers of chromatographic paper (soaked in 0.2 M ammonium acetate) were placed on the top of the membrane, and a stack of paper towels (5-8 cm) was placed on the top of the chromatographic papers. A glass plate with a weight (300-400 g) was placed on the top of the paper towels.

The DNA transfer was performed overnight (8-12 hours) at room temperature, and the paper towels were changed when they became wet. The blotting quality was controlled by staining the gel in a bath of ethidium bromide (0.5 μg/ml in water) for 45 minutes. After blotting DNA was fixed to the Hybond membrane under UV light for 40 seconds. The membrane is were hybridized immediately or wrapped in plastic and stored (dark) at 4° C.

1.3.5.2 Hybridization

Before hybridisation fixed membranes were prehybridised in a solution (10 ml) of 3×SSC, 0.1% SDS and 1.0% Blocking agent (Roche Molecular Biochemicals) for 2 hours at the selected hybridisation temperature for the different DNA probes (see above) in a Roller-Blot HB-3D hybridiser (Techne, Cambridge, UK). Biotinylated DNA-probes were then applied to the roller bottles in the hybridiser and incubated at 16-20 hours at the temperatures described for the different DNA-probes (ses above).

After hybridisation the membranes were washed 10 minutes in 50 ml 2×SSC−0.1% SDS, 10 minutes in 50 ml 0.1×SSC −0.1% SDS, and 10 minutes with PBS-T (all washes at room temperature). The membranes were incubated with Extravidin-Peroxydase (Sigma Chemical Co., St. Louis, Mo.), diluted 1:2000 i PBS-T for 30 minutes at room temperature (agitation), washed 2×10 minutes with PBS-T, 10 minutes with PBS (room temperature), and developed (10-20 minutes) in 30 ml PBS with 2 tablets diaminobenzidine (DAB; Sigma) and 24 ml 30% water-free H₂O₂. After development the membranes were rinsed in tap water and photographed.

1.4 Denaturing Gradient Gel Electrophoresis (DGGE)

1.4.1 Analytical DGGE

By DGGE PCR products were generated with general primers defining Bacteria (341fBAC and 907rBAC) or Archaea (21fARCH and 958rARCH). To the primers 341f BAC and 21fARCH a 40 mer GC-clamp was added to the 5′-end (5′-CGC-CCG-CCG-CGC-GCG-GCG-GGC-GGG-GCG-GGG-GCA-CGG-GGG-G-3′) (SEQ ID NO: 20).

DGGE was performed with 6% (w/v) polyacrylamide (PAA) gels in [0.5×] TAE buffer (20 mM Tris-acetat, pH 7.4; 10 mM acetat; 0.5 mM EDTA) with a 20-70% gradient of the denaturing agents urea and formamide (100% denaturing agents corresponded to 7 M urea and 40% (v/v) deionised formamide) in a DCode Universal Mutation Detection system (BioRad).

Stock solutions of PAA/Bis-acrylamide (Bis) (40%) consisted of 38.93 g acrylamide and 1.07 g Bis dissolved in deionised water to 100 ml, while stock solutions of [50×] TAE buffer was generated by mixing 242 g Tris, 57.1 g acetic acid, and 100 ml 0.5 M EDTA to a total volume of 1000 ml with deionised water. Linear gradient gels (thickness 1 mm) were prepared by mixing PAA and Bis with denaturating agents to generate a 20 to 70 g linear gradient in a gradient delivery system (BioRad modell 475). Solutions with 20% or 70% denaturing agents are described in Table 1 below:

TABLE 1
Composition of 20% and 70% denaturating
solutions used in DGGE

| | Denaturating Solutions | | |
|---|---|---|---|
| CHEMICALS | | 20% | 70% |

| | | | | |
|---|---|---|---|---|
| 40% acrylamide/Bis | 15 | ml | 15 | ml |
| [50×] TAE buffer | 2 | ml | 2 | ml |
| Formamide | 8 | ml | 28 | ml |
| Urea | 8.4 | g | 29.4 | g |
| Deionised water | to 100 | ml | to 100 | ml |

For the preparation of one gel 18 ml of each solution was mixed with 200 ml ammonium persulphate (10% (w/v) in deionised water) and 20 μl TEMED (BioRad), and the mixtures immediately transferred to each of two 30-ml syringes which were subsequently mounted in the gradient delivery system. The gel was cast as a parallel gradient gel (16×16 cm) with 1 mm thickness and allowed to polymerize for approximately 1 hour, and with a comb of 15 wells. The electrophoresis tank was filled with [1×] TAE buffer which was heated to 60° C. in the tank, and 1-2 polymerised gels placed vertically in the electrophoresis tank.

Each PCR product sample (10 μl) was mixed with 10 μl sample buffer (0.05% bromophenol blue, 0.05% xylene cyanol, 70% glycerol, diluted in deionised water), and the complete volume (20 μl) applied to each well.

Vertical electrophoresis was performed with continuous temperature (60° C.) and voltage (150 V) until both the markers had migrated to the bottom of the gel (approximately 4.5 hours).

After electrophoresis the gels were stained in SYBR Gold (Molecular Probes, Leiden, The Netherlands), diluted 1:10,000 in [1×] TAE. for 20-30 minutes. The gels were then photographed with the GelDoc system (BioRad).

The gel band patterns were compared for similarity and similarity indices generated by the Quantity One option of the GelDoc software program. The Dice Coefficient method was used, based on the following formula for similarity:

$$Similarity = 200 \times \frac{{\sum }_{i = 1}^{B} Min ( {s}_{i} , {t}_{i} )}{{\sum }_{i = 1}^{B} ( {s}_{i} + {t}_{i} )} ,$$

where S and T are vectors representing two lanes in the same band set that are being compared.
1.4.2 Preparative DGGE

Preparative DGGE was performed as described for the analytical DGGE, except that N,N′-bis-acrylylcystein (BAC; Sigma) was used instead of Bis during gel generation. The BAC enabled gel solution after electrphoresis (Muyzer et al., 1996).

PCR samples (300 μl) were precipitated with 30 μl of 5 M NaCl and 750 μl ethanol at −80° C. for 1 hour, centrifuged (20 000× g, 2 minutes) in a microcentrifuge (Eppendorf Model 5417C, Eppendorf, Hamburg, Germany). The pellet was washed with 70% ethanol, dried on a heating block (35° C., 20 minutes) and dissolved in 30 μl sterile water.

The gel was cast, samples applied, and electrophoresis run as described above.

The gel was stained after electrophoresis with SYBR Gold (see above), and selected bands cut under UV-illumination with sterile scalpels. Each slice of gel was transferred to a microcentrifuge tube, washed 2×10 minutes with 100 μl sterile water, and the water removed. β-mercaptoethanol (100 μl; BioRad) was applied and the tubes incubated for 16-20 hours at 37° C. Deionised water (100 μl), 0.1 volume 5 M NaCl, and 2.5 volumes ice cold ethanol was applied to each tube. The tubes were incubated at −80° C. for 2 hours, centrifuged (10 000× g, 20 minutes) in a microcentrifuge, and the supernatant removed carefully. The tubes were dried (35° C., 20 minutes), and the pellet was dissolved in 100 μl sterile water.

PCR product content in the samples was checked in PCR with primers defining Bacteria, but without GC-clamp. The PCR products were then purified in preparative agarose gel electrophoresis with low-melting temperature agarose (see above).

1.5 Cloning

1.5.1 TOPO TA Cloning

Cloning was performed with the TOPO TA Cloning kit (Invitrogen, Carlsbad, Calif., U.S.A), with the pCR 2.1-TOPO plasmid vector and One Shot TOP10 chemically competent Escherichia coli cells.

DNA was amplified in PCR using 341fBAC and 907rBAC primers defining Bacteria. PCR was performed as described above, except that the final termination at 72° C. was prolonged to 10 minutes to generate 3′-adenine overhangs. The PCR products were purified in preparative agarose gel electrophoresis with low-melting temperature agarose as described above. The gel slices with PCR products were melted as described (65° C., 15 minutes) and maintained melted at 37° until ligated into the vector.

Melted agarose slices (4 μl) were carefully mixed with 1 μl salt solution (1.2 M NaCl, 0.06 M MgCl₂) and 1 μl TOPO pCR 2.1 vector. The ligation was performed for 10 minutes at 37° C. The tubes were placed on ice, and transformation of the vector (4 μl) into chemically competent TOP10 cells (50 μl) performed on ice (15 minutes), followed by heat-shock (42° C., 30 seconds), and immediate transfer to ice (10 minutes). The transformation reaction was diluted in 250 μl SOC medium (2% Tryptone, 0.5% yeast extract, 10 mM NaCl, 2.5 mM KCl, 10 mM MgCl₂, 10 mM MgSO₄, 20 mM glucose), and the reaction in a shaking incubator at 37° C. for 1 hour with 200 rpm horizontal shaking.

The suspensions were spread on agar plate with Luria-Bertani (LB) agar medium (1.5% agar, 1.0% Tryptone, 0.5% yeast extract, 1.0% NaCl, pH 7.0; Sigma) supplemented with 50 μg/ml ampicillin or kanamycin antibiotics. Before inoculation the plates were spread with X-gal (5-bromo-4-chloro-3-indolyl b-D-galactopyranoside; Sigma), 40 μl of 40 mg/ml X-gal in dimethylformamide.

Suspensions (20 and 50 μl) of transformants were spread on the LB agar plates (20 μl suspensions were diluted with 20 μl SOC medium before plate spreading to ensure even spreading). The plates were incubated for 20-24 hours at 37° C. Only transformants with inserted plasmid grew on the medium, due to the resistance gene of the plasmid. Colonies with PCR products ligated into the vector were visualised as white colonies, as opposed to light or dark blue colonies with vectors without PCR product inserts. Discrete white colonies were isolated in liquid LB medium, supplemented with ampicillin or kanamycin (50 μg/ml). The medium was distributed in 24-well sterile tissue culture plates (Corning Inc., Corning, N.Y., USA), with 2 ml medium/well. The clones were incubated 20-24 hours in the LB medium, and the plasmids purified.

1.5.2 Isolation of Plasmids

The plasmids were isolated with a GenElute Plasmid Miniprep kit (Sigma), according to the instructions from the manufacturer. Transformant clones (2 ml) were harvested by centrifugatidn on a microcentrifuge Eppendorf) at 14 000× g for 2 minutes. The pellets were resuspended in a Resuspens ion Solution (200 μl) by vortexing. A Lysis Solution (200 μl) was added, the mixture gently inverted until clarification (8-10 times), and the lysis neutralized within 3-5 minutes with 350 μl Neutralization/Binding Buffer. The cell debris were pelleted by centrifugation (14 000× g for 10 minutes). The supernatants were transferred into GenElute Miniprep binding columns assembled into microcentrifuge tubes, centrifuged (14 000× g for 2 minutes), and the flow-through liquid discarded. The binding columns were then washed with 750 μl Wash Solution and centrifuged (14 000× g for 2 minutes), effluents discarded, and the columns re-centrifuged (14 000× g for 2 minutes) to remove any additional solutions. The binding columns were then transferred to new microcentrifuge tubes, 100 μl sterile water applied, and the tubes centrifuged (14 000× g for 2 minutes). The isolated plasmid solutions were stored at −20° C.

1.5.3 Control of Positive PCR Insert

Positive PCR inserts in the transformant vector was controlled by PCR with M13 primers defining vector sequences flanking the inserted sequence.

The primer sequences were:

(SEQ ID NO: 21)

M13 Forward primer (−20): 5′-GTA-AAA-CGA-CGG-CCA-
G-3′

(SEQ ID NO: 22)

M13 Reverse primer: 5′-CAG-GAA-ACA-GCT-ATG-AC-3′

The primer sites corresponded to the bases 391-406 (M13 Forward -20) and 205-221 (M13 Reverse) of the LacZa fragment of the vector. A plasmid without a positive PCR product insert would result in a 202 bp M13 PCR product, while a positive PCR product would result in a 769 bp PCR product.

A PCR mix was prepared as described above (see section 3.4.3) with the M13 primer set (50 μM stock solutions) Plasmid DNA template (2 μl) was diluted in PCR buffer and sterile water to a final volume of 90 μl as described above, mixed with PCR mix. The PCR was run according to the following sequence cycles:
Initial denaturation: 94° C. for 2 minutes
Denaturation: 95° C. for 1 minute
Primer annealing: 55° C. for 1 minute
DNA synthesis (primer extension): 72° C. for 1 minute
Number of cycles: 30

The PCR run was terminated at 72° C. (7 minutes) and cooling at 4° C.

1.5.4 Restriction Fragment Length Polymorphism (RFLP)

M13 PCR products with positive PCR product insert were analysed with the restriction endonucleases EcoR1 (Sigma), HaeIII (Sigma) and RsaI (Sigma). The restriction enzymes and corresponding enzymes (provided by the manufacturer; Sigma) is described in Table 2.

TABLE 2

Characteristics of restriction endonucleases
and their buffers

| | | RECOGNITION | DIGESTION | BUFFER COMP. |
|---|---|---|---|---|
| ENZYME | ^{A)} ACTIVITY | SEQUENCE | BUFFER | (1 × DILUTION) |
| EcoR | 40 000 U/ml | 5′ G/AATTC 3′ | Buffer SH | 50 mM Tris-HCl |
| | | | | 100 mM NaCl |
| | | | | 100 mM MgCl2 |
| | | | | 1 mM dithioerythritol |
| | | | | pH 7.5 |
| HaeIII | 10 000 U/ml | 5′ GG/CC 3 | Buffer SM | 10 mM Tris-HCl |
| | | | | 50 mM NaCl |
| | | | | 10 mM MgCl2 |
| | | | | 1 mM dithioerythritol |
| | | | | pH 7.5 |
| RsaI | 10 000 U/ml | 5′ GT/AC | Buffer SL | 10 mM Tris-HCl |
| | | | | 10 mM MgCl2 |
| | | | | 1 mM dithioerythritol |
| | | | | pH 7.5 |

A) One unit of each enzyme cleaves 1 mg 1 DNA in 1 hour at 37° C.

Plasmid DNA amplified by M13 PCR (1, 5, or 10 μl) were mixed with restriction enzymes: 1.0 μl EcoRI (40 U), 2.0 μl HaeIII (20 U), or 2.0 μl RsaI (20U). Each mixture was diluted to a total volume of 50 μl with the respective enzyme buffers [1×] concentration (see Table 2). The reaction mixtures were incubated at 37° C. for 2.5 hours and placed on ice to stop the reaction. The enzyme digestion was analysed as restriction fragment length polymorphism (RFLP) on analytical agarose gel electrophoresis.

1.6 Sequencing of Plasmid DNA

For sequencing purified plasmids were PCR amplified with the M13 primer set. DNA was measured by the ethidium bromide method, and precipitated with 60% ethanol and 0.1 M Na-acetate buffer (pH 4.6).

The precipitated PCR-products were submitted for DNA sequencing (MedProbe).

Sequences from the plasmids were submitted to the National Centre for Biotechnology Information (NCBI) using the BLAST program of the NCBI database (Altschul et al., 1997).

Phylogenetic trees and distance matrices were determined with the Phylip interphase of the Ribosomal Database Project (RDP; Maidak et al., 1997).

1.7 Detection of Bacteria and Archaea

The presence of Bacteria was detected by performing PCR using primers 341Bac and 907rBac. Following gel electrophoresis, the presence of a band of size 567 bp indicated the presence of bacteria in the sample (FIG. 1). The PCR products were transferred to Hybond N+ membrane by Southern blotting and subjected to hybridisation with various labelled probes e.g. for the δ subdivision bacteria including the SRB genera Desulphovibrio and Desulfobulbus (385-SRB). Both samples from one reservoir contained such bacteria, whereas the samples from the other did not (FIG. 2). The probe defining Desulfovibrio did not detect any of this species in the sample (not shown).

1.8 DNA Heterogeneity

1.8.1 DGGE

The genetic heterogeneity within the PCR products genrated above was investigated with DGGE analysis. PCR products defining Bacteria were concentrated 10 times and run with a 20-70% continuous gradient of denaturating agents. The results shown in FIG. 3 showed that the DNA profiles of the products from the samples varied considerably. Reservoir 1 well 1 showed 6 detected bands clustered in the lower area of the DGGE gel, while the other sample from this rservoir showed bands distributed over a large area. The two samples from the 2nd reservoir showed more homogeneous results, a total of 7-9 bands appeared in amplified DNA extracted from the water samples or emulsion phase.

The different bands were related to define melting conditions in the DGGE gel. The linear gradient generated in the gel was in the range 20-70% denaturing agent.

The diversity of the DGGE patterns were analysed by the Dice Coefficient method.

The similarity between samples from the same reservoir was >50%, ranging from 53.4 to 64.7% (average 58.6%). The similarity between samples from different reservoirs were <50% (range 22.2-33.5%, average 27.5%). The differences in similarity between samples from similar and different reservoirs were significant (P<0.05).

1.8.2 Cloning

A cloning strategy was used for the differentiation of the genetic variations in the reservoir samples. Bacterial PCR products were inserted into the vector pCR 2.1 and transformed into competent E. coli TOP 10 cells. After 3 separate clonings a number of the 101 clones with potential Bacteria PCR product inserts were analysed. Purified plasmids from the clones were analysed by M13 PCR, using primers defining plasmid sequences flanking the inserted PCR products. These primers were selected since they were specific for (annealed to) plasmid sequences flanking the inserted PCR products. In this way possible amplification of E. coli genomic 16S rDNA was avoided. After M13 PCR the products were visualised in agarose gel electrophoresis. Products with positive Bacteria PCR product inserts were detected as a band of approximately 800 bp, while an “empty” product (vector without any inserted PCR product) was visualised as a band of approximately 240 bp (data not shown).

The results showed that most analysed clones contained a positive Bacteria PCR insert (n=7/1), while a number showed no insert (n=21) and were excluded from further analysis. However, a few clones showed both products (n=9). This could be the result of transformation of more that 1 plasmid into individual clones, including copies both copies of PCR product insert and of “empty” plasmids.

Plasmids from 80 clones (clones containing PCR product inserts) were investigated further with the restriction endonucleases HaeIII and RsaI for restriction fragment length polymorphism (RFLP)-analysis. The plasmid sequences containing inserts were amplified (M13 PCR) and the products subjected to RFLP-analysis. After restriction enzyme digestion the patterns were visualised using agarose gel electrophoresis.

The RFLP analysis revealed a significant variation between the clones. No distinct differences were observed between clones with only positive PCR product inserts and clones with both positive and negative inserts, indicating that restriction fragments were restricted to the PCR product inserts. With HaeIII a total of 10 different genotypes were defined (A-J), and these are shown in FIG. 4, while RsaI restriction analysis revealed a total of 8 genotypes (a-h) as shown in FIG. 5. The base pair distributions of the digested PCR products are shown in Table 3 and 4.

TABLE 3
Base pair (bp) sizes of individual bands in FIG. 4 (HaeIII RFLP) as
determined by comparison to the known base sizes of the DNA standard

| | HaeIII RFLP type | | | | | | | | | |
|---|---|---|---|---|---|---|---|---|---|---|
| | A | B | C | D | E | F | G | H | I | J |
| base | 582 | 266 | 197 | 329 | 416 | 174 | 157 | 486 | 266 | 522 |
| pairs | 89 | 151 | 122 | 153 | 204 | 89 | 130 | 123 | 228 | 177 |
| | 70 | 126 | 89 | 112 | 89 | 70 | 106 | 89 | 112 | 112 |
| | | 89 | 70 | 89 | 70 | | 89 | 70 | 89 | 89 |
| | | 70 | | 70 | | | 70 | | 70 | 70 |

TABLE 4
Base pair (bp) sizes of individual bands in FIG.
5 (RsaI RFLP) as determined by comparison to
the known base sizes of the DNA standard

| | RsaI RFLP type | | | | | | | |
|---|---|---|---|---|---|---|---|---|
| | a | b | c | d | f | g | h | i |
| base pairs | 676 | 400 | 405 | 333 | 251 | 239 | 196 | 251 |
| | 92 | 244 | 239 | 239 | 157 | 199 | 167 | 198 |
| | 58 | 92 | 157 | 111 | 92 | 157 | 139 | 141 |
| | | 58 | 92 | 58 | 58 | 92 | 92 | 117 |
| | | | 58 | | | 58 | 58 | 58 |

The main genotype patterns exhibited minor or moderate differences with respect to the mobilities of individual bands. An example of this is shown for the HaeIII RFLP type A in FIG. 6. Corresponding variations appeared also for several of the other genotypes which contained several clones.

Comparison of the HaeIII and RsaI RFLP main types showed a relationship between the patterns generated by the two restriction enzymes. Clones exhibiting homologue pattern with HaeIII often exhibited homologue pattern with RsaI. A number of 22 unique types were defined based on the combined results with the two restriction enzymes, and among these 7 patterns dominated quantitatively with ≦5 clones. The relationship between RFLP patterns and reservoir origin is shown in Table 5. These results showed that most RFLP patterns were confined either to the one or to the other reservoir, and that individual well samples from each reservoir contained common RFLP types. Thus, the presence of a reservoir-specific rather than a well-specific microbial flora was indicated. A few RFLP types also appeared in both reservoirs (Table 5). However, closer examination, utilizing the high-resolution sub-type differences, revealed reservoir specificity also for these RFLP types, with one exception (Table 6). A HaeIII type A-B and RsaI type a-a appeared both in samples from reservoir 1 well 1 (1 clone) and reservoir 2 well 2 (2 clones).

Based on the relation between the HaeII and RsaI types a RFLP type system was generated which took advantage of the combined RFLP patterns generated by both restriction enzymes.

TABLE 5
Relationship between RFLP-types and reservoir origin

| | Reservoir origin | |
|---|---|---|
| | (no. clones) | |
| RFLP type | 1 | 2 |

| HaeIII | RsaI | 1 | 2 | 1 | 2 |
|---|---|---|---|---|---|
| A | a | | 1 | 1 | 6 |
| A | b | 2 | 1 | 2 | 3 |
| A | c | | | 1 | 1 |
| A | d | | 1 | | |
| A | e | | 1 | 1 | |
| B | c | 1 | | | |
| C | c | 1 | | | |
| D | b | 1 | 1 | | |
| D | c | 1 | | | |
| D | g | 4 | 1 | | |
| E | a | | | 1 | |
| E | c | 1 | | | |
| F | a | 1 | | | |
| G | a | 3 | 8 | | |
| G | b | 1 | | 10 | 9 |
| G | d | 2 | | 2 | 5 |
| H | b | 1 | | 1 | 1 |
| H | c | | | 1 | |
| H | e | | | | |
| H | g | | | | |
| I | h | | 1 | | |
| J | f | 1 | | | |

TABLE 6
Comparison of RFLP-types appearing in both reservoir samples

| | Reservoir origin | |
|---|---|---|
| | (no. clones) | |
| RFLP sub type | 1 | 2 |

| HaeIII | RsaI | 1 | 2 | 1 | 2 |
|---|---|---|---|---|---|
| A-A | a-a | | | 1 | 2 |
| A-B | a-a | | 1 | | 2 |
| A-C | a-a | | | | 1 |
| A-D | a-a | | | | 1 |
| A-A | b-a | | | 1 | 1 |
| A-B | b-a | | | 1 | |
| A-B | b-c | | | | 2 |
| A-D | b-d | 1 | | | |
| A-E | b-d | | 2 | | |
| A-A | e-c | | 1 | | |
| A-E | e-d | | | 1 | |
| H-A | b-a | | | 3 | 3 |
| H-A | b-c | | | 1 | |
| H-B | b-a | | | 6 | 6 |
| H-C | b-b | 1 | | | |

TABLE 7
Further analysis of RFLP types based on the combined patterns
of the HaeII and RsaI restriction analysis. All the clones apart
from clone 13 are specific to either reservoir 1 or 2. Reservoir
1 has oil producing zones in the temperature range 68-75° C.
and reservoir 2 has oil producing zones in the temperature range
82-90° C. It is clear that there is greater biodiversity
in the lower temperature reservoir.

| | | | | Reservoir origin | |
|---|---|---|---|---|---|
| RFLP type | HaeIII | RsaI | No. clones | 1 | 2 |
| 1 A | A-A | a-a | 3 | | 3 |
| 1 B | A-B | a-a | 3 | 1 | 2 |
| 1 C | A-C | a-a | 1 | | 1 |
| 1 D | A-D | a-a | 1 | | 1 |
| 2 A | A-A | b-a | 2 | | 2 |
| 2 B | A-A | b-c | 1 | | 1 |
| 2 C | A-B | b-a | 1 | | 1 |
| 2 D | A-B | b-c | 1 | | 1 |
| 2 E | A-D | b-d | 1 | 1 | |
| 2 F | A-E | b-d | 2 | 2 | |
| 3 A | A-A | c-g | 1 | | 1 |
| 3 B | A-F | c-f | 1 | | 1 |
| 4 | A-A | d-a | 1 | 1 | |
| 5 A | A-A | e-c | 1 | 1 | |
| 5 B | A-E | e-d | 1 | | 1 |
| 6 | B | c-a | 1 | 1 | |
| 7 | C | c-a | 1 | 1 | |
| 8 A | D-B | b-c | 1 | 1 | |
| 8 B | D-D | b-a | 1 | 1 | |
| 9 | D-A | c-b | 1 | 1 | |
| 10 A | D-A | g | 3 | 3 | |
| 10 B | D-B | g | 1 | 1 | |
| 10 C | D-C | g | 1 | 1 | |
| 11 | E-A | a-a | 1 | | 1 |
| 12 | E-A | c-e | 1 | 1 | |
| 13 | F-A | a-a | 1 | 1 | |
| 14 A | G-A | a-a | 5 | 5 | |
| 14 B | G-B | a-d | 1 | 1 | |
| 14 C | G-B | a-f | 1 | 1 | |
| 14 D | G-C | a-b | 2 | 2 | |
| 14 E | G-D | a-b | 1 | 1 | |
| 14 F | G-E | a-c | 1 | 1 | |
| 15 | G-B | b-a | 1 | 1 | |
| 16 | G-A | d-b | 1 | 1 | |
| 17 A | H-A | b-a | 6 | | 6 |
| 17 B | H-A | b-c | 1 | | 1 |
| 17 C | H-B | b-a | 12 | | 12 |
| 17 D | H-C | b-b | 1 | 1 | |
| 18 A | H-A | c-b | 1 | | 1 |
| 18 B | H-A | c-c | 4 | | 4 |
| 18 C | H-A | c-d | 1 | | 1 |
| 18 D | H-A | c-h | 1 | | 1 |
| 19 A | H-B | e-b | 1 | | 1 |
| 19 B | H-E | e-a | 1 | | 1 |
| 20 | H-B | g | 1 | | 1 |
| 21 | I | h | 1 | 1 | |
| 22 | J | f | 1 | 1 | |

1.9 Sequencing

A selection of clones representing unique RFLP types were sequenced with a commercial Primer Walking Service (MedProbe). The sequencing analysis were performed on Bacteria PCR products generated plasmids containing 16S rDNA PCR product inserts, with an M13 primer set which included plasmid regions flanking the inserted products. The Primer Walking Service included a single strand sequencing of both M13-amplified strands from the 5′-starting nucleotide of each strand.

A number of sequences were BLAST, analysed by the NCBI database, or by the Ribosomal Database Project. For the clones with complete sequences identified the whole 16S rDNA product (positions 341 to 907 of the gene) was used for BLAST analyses, while only parts of the product was used for the clones with incomplete sequences. In this way a variety of different microorganisms were identified.

### EXAMPLE 2

Sample Materials

Samples of “fines” were received from Statoil in several batches.

The samples were of the following types: water, water/oil, dry filtered, oil (toluene phase), emulsion phase, washed emulsion, fibre in toluene, fibre in water.

Extraction of Nucleic Acids

Different approaches were used for nucleic acid extraction.

Phenol-Chloroform-Isoamylalcohol Extraction

Some samples were extracted according to this method:
Samples received were immediately filtered through Durapore filters (exclusion limit 0.22 μm), and the filters were stored in 2 ml lysis buffer (50 mM Tris-HCl, pH 8.0; 40 mM EDTA; 750 mM sucrose) at −20° until DNA extraction.

Extraction of frozen filters were performed as follows:
“Fine” materials were thawed and lysed directly on the filters. Lysis was performed by incubation of each filter with 2 μg lysozyme (from a 20 mg/ml stock solution; 37° for 30 minutes), followed by incubation with 1 μg Proteinase K (from a 20 mg/ml stock'solution) and 1% (w/v) sodium-dodecyl sulphate (SDS; from a 20% stock solution) at 55° C. for 2 hours.

The lysates were treated and analysed as in Example 1.

Extraction with a Commercial DNA Extraction Kit

Nucleic acids from other samples were extracted by a simplified method using the commercial kit “Genomic Prep DNA Isolation Kit” (Pharmacia Biotech, Uppsala, Sverige).

Some samples were filtered through Durapore filters, and the filters placed in 1 ml of “cell lysis reagent” of the genomic prep kit. The dry filter samples were crushed and incubated in 1 ml of “cell lysis reagent”. The rest of the samples were centrifuged (16000× g; 5 minutes), the supernatant discarded, and the pellet suspended in 0.6 ml “cell lysis reagent”. Suspensions were incubated at 80° C. for 10 minutes for cell lysis, cooled to room temperature, 3 μl “RNase A Solution” applied and the sample incubated at 37° C. for 30 minutes. “Protein Precipitation Solution” (200 μl) was applied, tubes vigorously shaken (20 seconds) for protein precipitation and centrifuged (15000× g; 5 minutes). Supematants (600 μl) were then mixed with 100% isoproanol (600 μl), mixed, centrifuged (1500× g; 3 minutes), the pellet washed with 600 μl ethanol (70% (v/v) and centrifuged (15000× g; 3 minutes). Pellets were dissolved in 100 μl “DNA Hydration Solution” and incubated at room temperature over night. Solutions of extracted nucleic acids were stored at −20°.

Polymerase Chain Reaction (PCR) Amplification

Oligonucleotides and deoxynucleotides were as described in Example 1 and Touchdown PCR was also as described in Example 1.

Analytical agarose gel electrophoresis or DGGE was then carried out as in Example 1.

Results and Discussions

Detection of Bacteria and Archaea by PCR

A selection of the extracted material was subjected to PCR amplification by primer sets defining Bacteria or Archaea. Most of the samples described were tested in PCR with both primer set.

The results of the testing could be separated in three; a) water/emulsion phase “fines”, b) oil phase “fines” (toluene extracts), and c) results from dry filters. The PCR results with bacterial primer set from the water or emulsion phases were positive for 14 of 20 tested samples (70%), for the oil phase 9 of 11 (82%) and for dry filters all 7 tested samples were strongly positive. All positive PCR products showed single DNA bands of the expected size of approximately 570 bp. The PCR results with the archaeal primers showed multiple bands of weak to moderate intensity for of 8 of 35 tested samples. None of these bands were of the expected size of 930 bp. We therefore suggest that the archaeal bands were false negative results, generated by some spurious primer binding. Some of the PCR-products showed very strong bands in agarose gel electrophoresis. These were confined to water-samples or to samples originating from dry filters.

The detection of positive PCR-products in the “fines” recovered from the oil phase by toluene extraction has not been reported before. It may be speculated if the “fines” showed amphiphatic characteristics. The bacteria may “hide” within water-containing and protective polymeric cap that may penetrate into the oil phase. In the oil phase, the outside of the “fines” may be mainly of a hydrophobic character while the interior is hydrophilic, capturing water and protecting the microbes. This raises questions whether microbes may reside and flourish within polymeric caps in oil phases devoid of water. Thus, the microbes may survive simply in water-containing polymer caps.

The PCR products with bacterial primers showed variable results related to the individial series. Most samples were extracted by a simple DNA extraction method (commercial Genomic Prep DNA kit), showing variable PCR results. After DNA extraction with the commercial kit residues of oil could be visualised in several extracts, both in water/oil and with toluene preparations, but not in extracts from dry filters. However, the first series of samples were extracted with the phenol-chloroform-isoamylalcohol method.

With this method the PCR products showed excellent band intensity in agarose gel electrophoresis, and no traces of oil were observed in the DNA extracts. Thus the PCR-results could be related to inhibitory oil compounds remaining in the oil after extraction. The main reason for not employing the phenol-chloroform-isoamylalkohol method on all samples was the labour intensity of this method. The results therefore emphasised the requirements for optimising DNA extraction procedures.

A modified phenol-chloroform-isoamylalkohol method will enable the extraction of larger numbers of samples.

In conclusion bacteria were detected in samples recovered both from most samples tested, including both water/emulsions/oil phases, oil-phases (recovered in toluene), and dry filters of special interest was the detection of bacterial DNA in the oil-phase samples. The culture-independent methods used here enabled microbial analysis despite destructive sample pre-treatment, which resulted in killing of the microbes present in the samples.

DGGE Analysis

DGGE analyses of a selection of the samples were performed with PCR products amplified with bacterial primer set. The results are visualised in FIG. 7 and summarised in Table 7.

TABLE 8
A summary of the DGGE results described in FIG. 7.

| | | | | ^{A)}Band mi- |
|---|---|---|---|---|
| | | | | gration |
| | | Received | Bands | (relative |
| Samples | Type of Sample | Dates | (no.) | front) |
| 90B | water | 14.08.01 | (1) | 73 |
| A-36 | water | 14.08.01 | 1 | 74 |
| A-14 | water/oil | 14.08.01 | 6 | 37, 52*, 64, |
| | | | | 70, 78* |
| A-13 | water/oil | 14.08.01 | 6 | 34, 45, 52*, |
| | | | | 73, 77* |
| A-48 | water | 14.08.01 | 2 | 50, 73 |
| A-09B | water (not filtered) | 14.08.01 | 2 | 27, 38, 51* |
| A-14 | water (not filtered) | 14.08.01 | 3 | 26, 36, 49* |
| HD-A-09B | Dry filter | 14.08.01 | 3 | 26, 36, 49* |
| 1 | oil (toluene phase) | 28.09.01 | 1 | 69 |
| 2 | oil (toluene phase) | 28.09.01 | 2 | 48, 69 |
| 3 | oil (toluene phase) | 28.09.01 | 1 | 69 |
| 4 | oil (toluene phase) | 28.09.01 | 0 | — |
| 5 | oil (toluene phase) | 28.09.01 | 1 | 66 |
| 6 | oil (toluene phase) | 28.09.01 | 1 | 69 |
| 7 | oil (toluene phase) | 28.09.01 | 1 | 69 |
| A-52 | emulsion phase | 28.09.01 | 1 | 69 |
| A-09B | emulsion | 28.09.01 | (1) | 66 |
| A-09 | washed emulsion | 28.09.01 | 1 | 68 |
| A-36 | emulsion | no infor- | 4 | 41, 54, 64*, |
| | | mation | | 66*, 68* |
| 1 | dryfilter | 23.10.01 | 2 | 49, 72 |
| 2 | dryfilter | 23.10.01 | 2 | 49, 71 |
| 3 | dryfilter | 23.10.01 | 4 | 49, 56, 63*, |
| | | | | 72* |
| 4 | dryfilter | 23.10.01 | 5 | 48, 56, 63, |
| | | | | 66*, 71 |
| 5 | dryfilter | 23.10.01 | 6 | 27, 29, 51, |
| | | | | 56, 61*, 64*, |
| | | | | 71* |
| 6 | dryfilter | 23.10.01 | 2 | 48, 70 |
| A-36 (1) | oil/emulsion/water | 14.11.01 | 2 | 48, 78 |
| A-36 (2) | oil/emulsion/water | 14.11.01 | 2 | 48, 71 |
| A-09B (3) | oil/emulsion/water | 14.11.01 | 2 | 49, 70 |

^{A)}The numbers marked with asterisk were those visually considered to be the main DGGE bands by intensity

### EXAMPLE 3

Correlation of bacterial species present with different wells in field 2.

The methods described in Examples 1 and 2 were used to study the diversity of microbial populations in 3 different wells in field 2.

FIG. 8 shows the DGGE bands seen when DGGE was performed on PCR products generated using the bacteria specific probes outlined above. The fact that different DGGE banding is seen for the three wells indicates that the microbiological profile overall may be different. This was investigated further by cloning and sequencing the PCR products as described in Examples 1 and 2. The pattern seen with simply performing DGGE can give an indication of the microorganisms present (for example A14 gives a typical pattern of Arcobacter).

The results of the cloning and sequencing are shown in FIG. 9, which shows the number of clones of the various species present in the three wells. Only α proteobacteria are found in more than one well.

This shows that mapping of a sample to an individual sample well, within a reservoir is possible. This may also be linked further to particular reservoir zones or with a particular mineralogical oil type. The well may also be surveyed with respect to its production and/or the data could be linked to the temperature range of the particular reservoir zone.

### EXAMPLE 4

The RFLP type of Archaea was investigated in samples taken from three different North-Sea seeps. The methods were carried out as above and the results further substantiate the fact that different microbiological profiles can be correlated with samples being taken from different locations. For example RFLP type I is found at all 3 locations whereas RFLP types 3, 6 and 7 are each found at only one location (FIG. 10).

1. A method of detecting, characterising or monitoring a hydrocarbon zone, which method comprises the genotypic analysis of a sample for the presence of one or more thermophilic or extremophilic microorganisms.

2. A method as claimed in claim I wherein the sample is from a sub-surface formation.

3. A method as claimed in claim 1 wherein the sample is oil, water or a oil/water mixture from an exploration or production well.

4. A method as claimed in claim 1 wherein the sample is oil or water that has been exposed to oil.

5. A method as claimed in claim 1 wherein information regarding the microorganisms present is utilised in an ongoing exploration or production process.

6. A method as claimed in claim 1 wherein a plurality of different microorganisms are detected thereby generating a microbiological profile for said sample.

7. A method as claimed in claim 6 wherein the generated microbiological profile is compared against one or more reference profiles.

8. A method as claimed in claim 1 wherein the sample is analysed for the presence of Archaeglobus, Erythrobacter, Arcobacter, Geothermobacter, Thermodesulforamonas and Thermotogales.

9. A method as claimed in claim 1 which does not comprise a culturing step.

10. A method as claimed in claim 1 wherein the sample is contacted with one or more different oligonucleotides designed to hybridise to regions of nucleic acid from or derived from the thermophilic or extremophilic microorganisms.

11. A method as claimed in claim 1 wherein at least some of the nucleic acid within the sample is amplified.

12. A method as claimed in claim 1 wherein the hydrocarbon zone is characterised such that information about the type of oil, the quantity of oil, the quality of the oil, the sulphur content of the oil, the presence of gas or the gas:oil ratio is obtained.

13. A method as claimed in claim 1 wherein the hydrocarbon zone is characterized such that the depth of a hydrocarbon zone is determined, wherein the particular microorganisms identified are indicative of a certain depth.

14. A method as claimed in claim 1 wherein the hydrocarbon zone is characterised such that the migration route of said hydrocarbon zone is determined.

15. A method as claimed in claim 13 wherein the hydrocarbon zone is an oil reservoir.

16. A method as claimed in claim 1 wherein genotypic analysis is performed using one or more probes selected from the group consisting of SEQ ID No. 1 to SEQ ID No. 19.

17. The use of one or more oligonucleotide probes preferably a battery of probes in the generation of a microbiological profile of a sample as defined in claim 2, wherein said profile is for detecting, characterizing or monitoring a hydrocarbon zone.

18. The use of claim 17 wherein said microbiological profile is a pattern which can be compared with a reference sample.

19. A battery of probes for use in the method of claim 1.

20. An oligonucleotide as defined in any one of SEQ ID No. 5 to SEQ ID No. 19 or a functionally equivalent variant thereof.

21. A solid support having attached thereto one or more oligonucleotides as defined in claim 20.

22. A solid support as claimed in claim 21 which is a microchip.

23. A solid support having attached thereto one or more oligonucleotides as defined any one of SEQ ID No. 5 to SEQ ID No. 19 or a functionally equivalent variant thereof, wherein the sold support has at least 4 oligonucleotides attached thereto.

24. A kit for use in a method as claimed in claim 1 comprising one or more oligonucleotides as defined in any one of SEQ ID No. 5 to SEQ ID No. 19 or a functionally equivalent variant thereof.
